(19) United States
(12) Patent Application Publication
(10) Pub. No.: US 2022/0125436 A1
(43) Pub. Date: Apr. 28, 2022

(54) DEVICES, SYSTEMS, AND METHODS FOR MINIMALLY INVASIVE MODULATION OF ABDOMINAL TISSUE

(71) Applicant: Vasu Sunkara, Anaheim, CA (US)

(72) Inventor: Vasu Sunkara, Anaheim, CA (US)

(21) Appl. No.: 17/448,036

(22) Filed: Sep. 17, 2021

Related U.S. Application Data
(60) Provisional application No. 63/079,980, filed on Sep. 17, 2020.

(51) Int. Cl. A61B 17/12; A61B 18/14

(52) U.S. Cl. CPC A61B 17/12099; A61B 17/12168; A61B 17/12186; A61B 2018/00577; A61B 18/1482; A61B 17/12031; A61B 17/1214

(57) ABSTRACT

Devices, systems, and methods for treating the pancreas are disclosed herein. For example, some aspects of the technology are directed to a method for treating a medical condition affecting the pancreas of a human patient, wherein the method comprises implanting an occlusive device within the pancreatic duct to induce atrophy of pancreatic tissue.

### CROSS-REFERENCE TO RELATED APPLICATIONS

The present application claims the benefit of priority to U.S. Patent Application No. 63/079,980, filed Sep. 17, 2020, which is incorporated herein by reference in its entirety.

The present application incorporates by reference PCT Application No. PCT/US20/22408, filed Mar. 12, 2020, in its entirety.

### TECHNICAL FIELD

The present technology relates to devices, systems, and methods for modulating abdominal tissue. In some embodiments, the present technology is directed to devices, systems, and methods for modulating pancreatic tissue.

### BACKGROUND

Pancreatitis and pancreatic cancer are two types of pathology in the human pancreas that engender serious human morbidity and mortality and have limited treatment options.

Pancreatitis is the inflammation of pancreatic tissue and can present in acute, acute relapsing, and chronic states. The predominant symptom of chronic pancreatitis is severe, chronic abdominal pain due to progressive and irreversible morphological changes that permanently impair pancreatic function. While the pathogenesis of chronic pancreatitis remains poorly understood, one proposed etiology is the overactive intrapancreatic activation of digestive acinar enzymes.

Pancreatic cancer is one of the most lethal human neoplasms. For all stages of pancreatic cancer combined, the one-year relative survival rate is 20%, and the five-year rate is 7%. Because of its non-specific symptoms and the lack of consensus on effective screening guidelines, pancreatic cancer is too often identified late in its course when there are limited treatment options. As a result, it has one of the lowest survival rates of any human cancer. The most common type of pancreatic cancer, adenocarcinoma of the pancreas, begin in the exocrine cells of the pancreas, which make up 95% of the pancreas.

In general, the most viable treatment options for chronic pancreatitis and early-stage pancreatic cancer remain limited to surgical approaches. (For late-stage pancreatic cancer, no curative therapies currently exist.) A partial or total pancreatectomy (i.e., removal of the pancreas) is one such surgical approach that has been shown to be effective in treating pancreatic disease. However, removal of pancreatic tissue comes with a host of risks and complications, such as an increased risk of exocrine insufficiency and diabetes.

Another surgical approach proposed by at least one research group in the 1960's and 70's is pancreatic duct ligation. Though this method gained little traction and is much less known and practiced than other surgical methods, the researchers were able to demonstrate the therapeutic value of surgical pancreatic duct ligation in the treatment of pancreatitis. In particular, researchers showed that surgical ligation of the pancreatic duct induced atrophy of the pancreatic exocrine cells, which led to functional and physiologic improvements for the patient, including a reduction in serum amylase levels. A minimally invasive form of pancreatic duct ligation has recently been proposed that comprises injecting a composition directly into the lumen of the pancreatic duct to decrease the secretion of digestive enzymes.

In light of the foregoing, a need exists for improved devices and methods for minimally invasive treatment of the pancreas.

### SUMMARY

The subject technology is illustrated and described according to various aspects described below, including with reference to FIGS. 2-24B. Various examples of aspects of the subject technology are described as numbered clauses (1, 2, 3, etc.) for convenience. These are provided as examples and do not limit the subject technology.

Clause 1. A method for treating a medical condition affecting the pancreas of a human patient, the pancreas including a pancreatic duct having a lumen extending therethrough, the method comprising implanting an occlusive device within the pancreatic duct to induce atrophy of pancreatic tissue.

Clause 2. A method for treating a medical condition affecting the pancreas of a human patient, the pancreas including a pancreatic duct having a lumen extending therethrough, the method comprising:
endoluminally delivering a distal portion of a treatment device proximate the pancreatic duct, wherein the treatment device comprises (a) an elongated member having a distal region configured to navigate the lumen of the duct, and (b) a treatment element carried by a distal portion of the elongated member;
deploying the treatment element within the pancreatic duct lumen to form an obstruction within the pancreatic duct lumen;
detaching the elongated member from the treatment element such that the treatment element is implanted within the pancreatic duct lumen; and
withdrawing the elongated member from the patient.

Clause 3. The method of Clause 1, wherein the obstruction induces atrophy of pancreatic tissue.

Clause 4. The method of any one of the previous Clauses, wherein the obstruction induces atrophy of pancreatic exocrine tissue.

Clause 5. The method of any one of the previous Clauses, wherein the obstruction substantially prevents exocrine duct secretions from entering the pancreatic duct lumen from one or more adjacent exocrine ducts.

Clause 6. The method of any one of the previous Clauses, wherein the obstruction substantially prevents exocrine duct secretions from traveling proximally beyond the treatment element.

Clause 7. The method of any one of the previous Clauses, wherein deploying the treatment element includes expanding the treatment element into apposition with an inner surface of the pancreatic duct wall such that the treatment element obstructs at least a portion of the cross-sectional area of the pancreatic duct lumen.

Clause 8. The method of any one of the previous Clauses, wherein deploying the treatment element includes expanding the treatment element into apposition with an inner surface of the pancreatic duct wall such that the treatment element obstructs substantially all of the cross-sectional area of the pancreatic duct lumen.

Clause 9. The method of any one of the previous Clauses, wherein deploying the treatment element includes expanding the treatment element into apposition with an inner surface of the pancreatic duct wall such that the treatment element obstructs less than all of the cross-sectional area of the pancreatic duct lumen.

Clause 10. The method of any one of the previous Clauses, wherein the pancreatic duct is the main pancreatic duct.

Clause 11. The method of any one of the previous Clauses, wherein the pancreatic duct is the accessory pancreatic duct.

Clause 12. The method of any one of the previous Clauses, wherein deploying the treatment element creates an obstruction within a lumen of the main pancreatic duct and within a lumen of the accessory pancreatic duct.

Clause 13. The method of any one of the previous Clauses, wherein the treatment element is configured to be positioned within the pancreatic duct lumen such that the treatment element extends along substantially the entire length of the main pancreatic duct, the entire length of the accessory pancreatic duct, or both.

Clause 14. The method of any one of the previous Clauses, wherein the treatment element is configured to be positioned within the pancreatic duct lumen such that the treatment element extends along less than the entire length of the main pancreatic duct, the entire length of the accessory pancreatic duct, or both.

Clause 15. The method of any one of the previous Clauses, wherein the treatment element is configured to be positioned within the pancreatic duct lumen such that the treatment element extends along less than an entire length of the main pancreatic duct, less than an entire length of the accessory pancreatic duct, or both.

Clause 16. The method of any one of the previous Clauses, wherein the treatment element is a first treatment element and the obstruction is a first obstruction, and wherein the method further comprises deploying a second treatment element within the pancreatic duct lumen to form a second obstruction.

Clause 17. The method of Clause 14, wherein the first and second treatment elements are expandable, occlusive devices.

Clause 18. The method of Clause 14, wherein the first obstruction is a body of the treatment element in an expanded state and the second treatment element is configured to deliver energy to a wall of the pancreatic duct to create a second obstruction in the form of scar tissue.

Clause 19. The method of any one of the previous Clauses, wherein the treatment element creates the obstruction by ablating a portion of a wall of the pancreatic duct.

Clause 20. The method of any one of the previous Clauses, wherein the treatment element creates the obstruction by ablating a portion of a wall of the pancreatic duct via radiofrequency ablation.

Clause 21. The method of any one of the previous Clauses, wherein the treatment element creates the obstruction by ablating a portion of a wall of the pancreatic duct via cryoablation.

Clause 22. The method of any one of the preceding Clauses, wherein the treatment element comprises at least one of a mesh, a stent, a coil, a balloon, a liquid embolic, a foam, a glue, or a hydrogel.

Clause 23. The method of any one of the previous Clauses, wherein the treatment device is delivered through the digestive tract.

Clause 24. The method of any one of the previous Clauses, wherein the treatment device is delivered through an elongated shaft.

Clause 25. The method of any one of the previous Clauses, wherein the treatment device is delivered through an endoscope.

Clause 26. The method of any one of the previous Clauses, wherein the medical condition is chronic pancreatitis.

Clause 27. The method of any one of the previous Clauses, wherein the medical condition is acute pancreatitis.

Clause 28. The method of any one of the previous Clauses, wherein the medical condition is acute relapsing pancreatitis.

Clause 29. The method of any one of the previous Clauses, wherein the medical condition is pancreatic cancer.

Clause 30. A method for treating a medical condition affecting the pancreas of a human patient, the pancreas including a pancreatic duct having a lumen extending therethrough, the method comprising:
endoluminally delivering an occlusion element to the pancreatic duct lumen;
implanting the occlusion device within the pancreatic duct lumen;
with the occlusion device, (a) preventing exocrine duct secretions from entering the pancreatic duct lumen, (b) traveling proximally beyond the treatment element, or (c) both (a) and (b); and
inducing atrophy of pancreatic tissue.

Clause 31. A device for treating a medical condition affecting the pancreas of a human patient, the pancreas including a pancreatic duct having a lumen extending therethrough, the device comprising:

an occlusion element configured to be implanted within a lumen of the pancreatic duct to block exocrine duct secretions from entering the pancreatic duct lumen and/or to prevent exocrine duct secretions from exiting the pancreatic duct.

Clause 32. The device of Clause 29, wherein the treatment element comprises at least one of a mesh, a stent, a coil, a balloon, a liquid embolic, a foam, a glue, or a hydrogel.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic view of a human pancreas in accordance with the present technology.

FIG. 2 shows a system for modulating the pancreas in accordance with the present technology.

FIG. 3A is an enlarged view of a distal portion of a treatment system in accordance with the present technology.

FIG. 3B is an end view of the distal portion shown in FIG. 3A.

FIG. 4A is an enlarged view of a distal portion of a treatment system in accordance with the present technology.

FIG. 4B is a cross-sectional end view of the distal portion shown in FIG. 3A, taken along line 4B-4B.

FIGS. 5A-5C depict a method for ligating the pancreatic duct using a treatment system of the present technology.

FIG. 6 depicts a method for ligating the pancreatic duct using a treatment system of the present technology that includes an elongated shaft with a lateral distal opening.

FIGS. 7A-10 show enlarged, isolated views of distal portions of treatment devices having different treatment elements. In FIGS. 7A-10, the treatment elements are shown in their respective deployed configurations.

FIGS. 11A-11C illustrate a method for creating scar tissue at the pancreatic duct using the treatment devices of the present technology.

FIGS. 12-14 illustrate different methods for ligating the pancreatic duct via a laparoscopic approach in accordance with the present technology.

FIGS. 15A-15P illustrate different catheters in accordance with the present technology.

FIG. 16A illustrates the arteries associated with the pancreas.

FIG. 16B illustrates the veins associated with the pancreas.

FIG. 17 illustrates the nerves responsible for innervating the pancreas.

FIG. 18A shows a treatment device for modulating the pancreas in accordance with the present technology.

FIGS. 18B and 18C are enlarged views of a distal portion of the treatment device shown in FIG. 18A.

FIG. 18D is a treatment element for use with the treatment devices and systems of the present technology.

FIG. 18E shows a portion of the treatment device of FIG. 18A.

FIG. 19 depicts a method for ligating the pancreatic duct using the treatment device shown in FIGS. 18A-18E.

FIG. 20 depicts a portion of a treatment system for modulating the pancreas in accordance with the present technology.

FIGS. 21A and 21B illustrate the motion of a conveyor system configured in accordance with the present technology.

FIG. 22 shows a treatment device configured in accordance with the present technology.

FIG. 23 is a partial cross-sectional view of a treatment device for modulating the pancreas in accordance with the present technology.

FIG. 24A shows a system for modulating the pancreas in accordance with the present technology.

FIG. 24B is an enlarged view of a distal portion of the treatment system shown in FIG. 24A.

### DETAILED DESCRIPTION

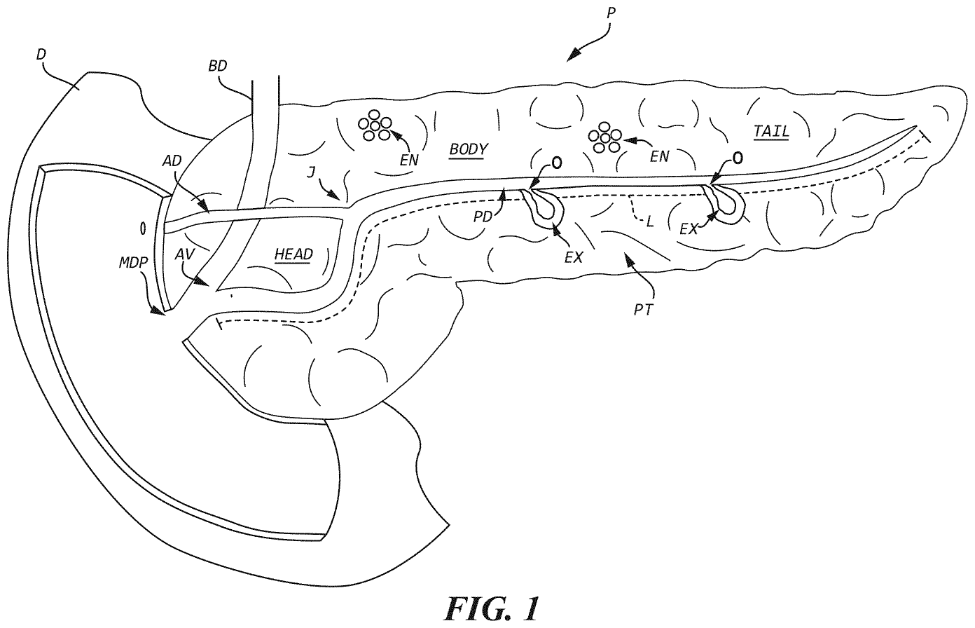

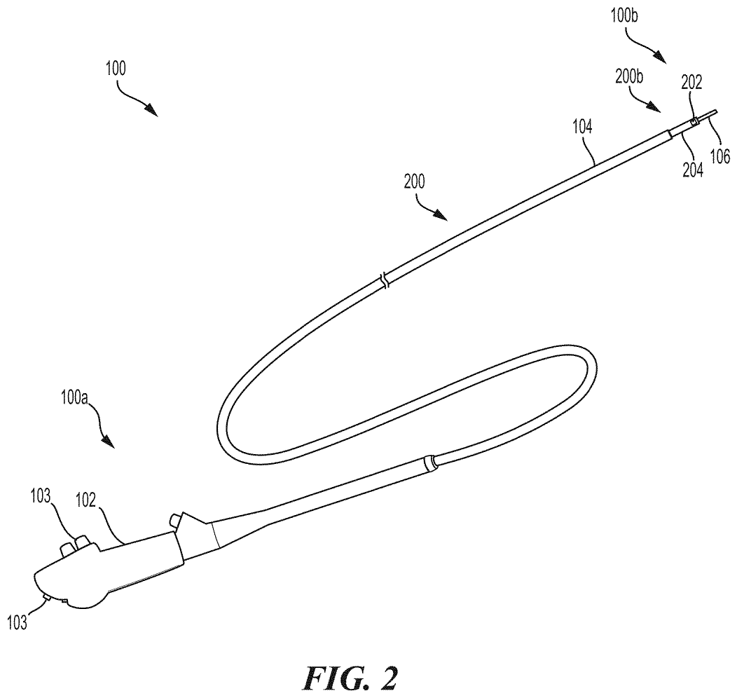

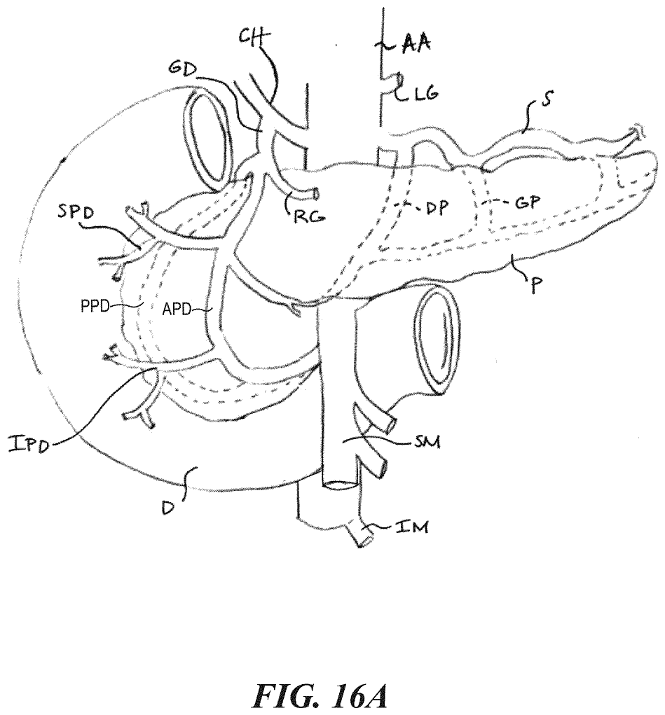

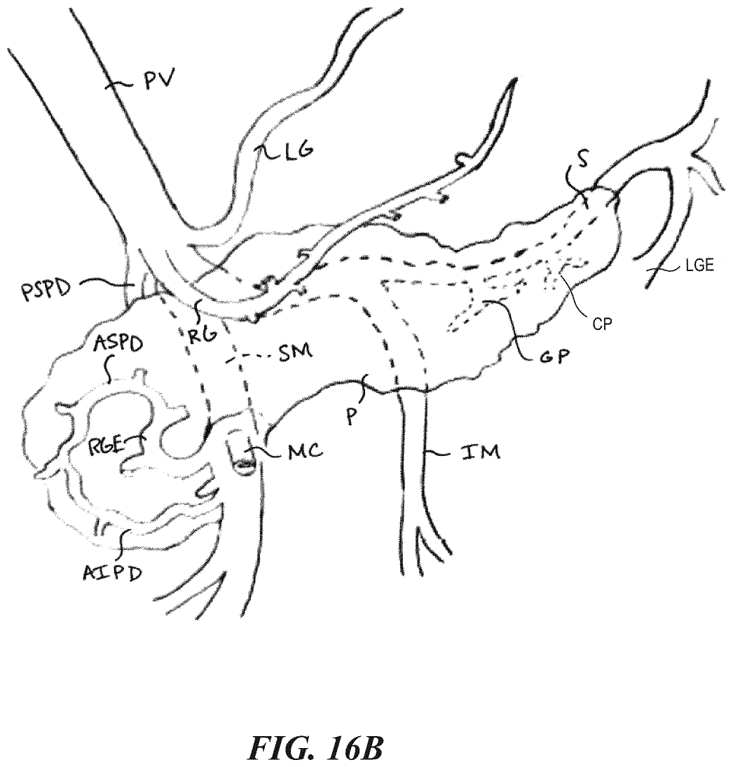

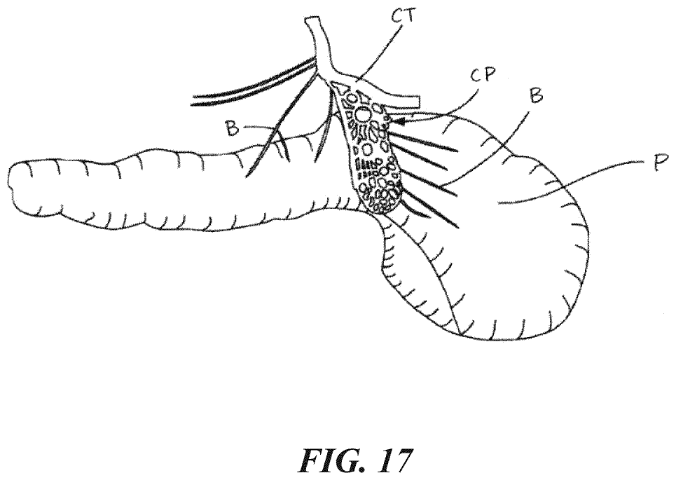

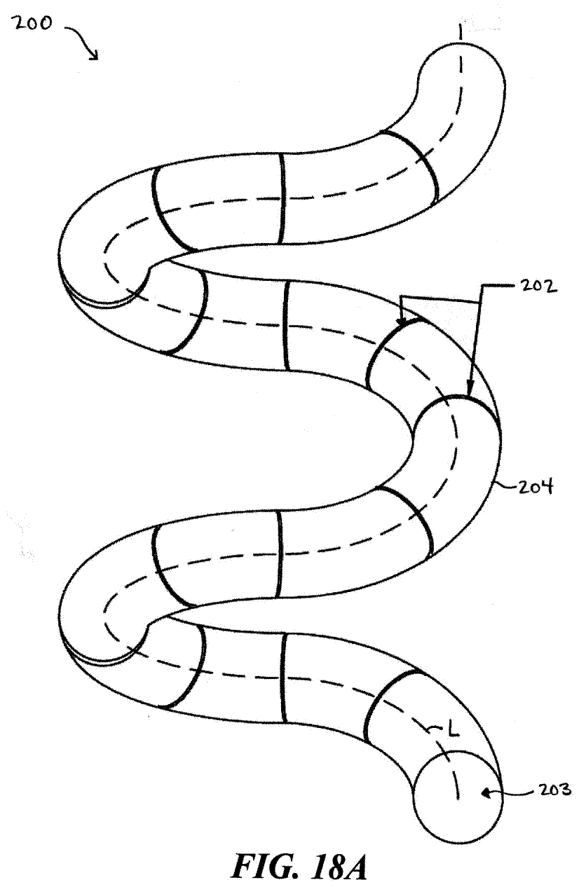

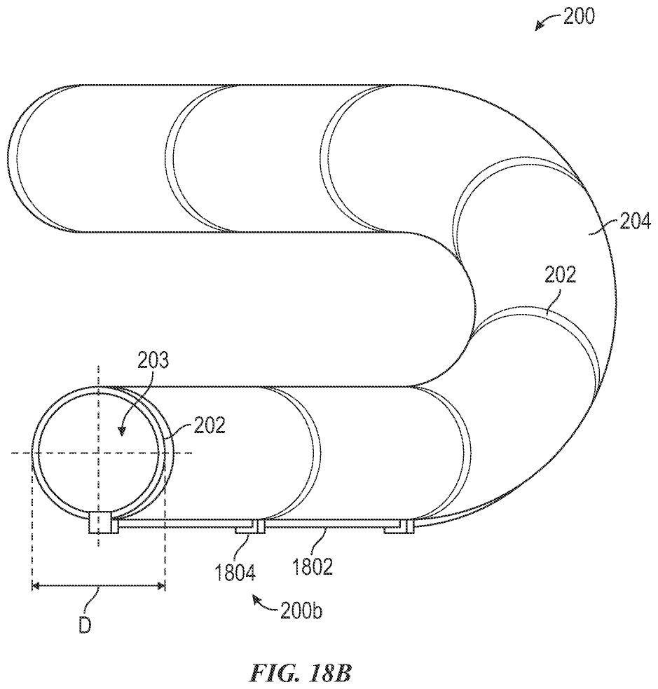

The present technology relates to devices, systems, and methods for modulating retroperitoneal tissue, such as the pancreatic tissue. Selected definitions are provided in Section I. An overview of the pancreatic anatomy and physiology referenced throughout is provided below with reference to FIG. 1 and Section II. Selected examples of devices, systems, and methods for pancreatic duct ligation are described below with reference to FIGS. 2-15 and 18A-24B and Sections III, IV, and VII. Selected examples of devices, systems, and methods for modulating pancreatic tissue by affecting the blood supply to the pancreas are described below with reference to FIGS. 16A-16B and Section V. Selected examples of devices, systems, and methods for modulating pancreatic tissue by modulating one or more nerves innervating pancreatic tissue are described below with reference to FIG. 17 and Section VI.

### I. Definitions

As used herein, “the pancreatic duct” refers to all or a portion of the main pancreatic duct and/or all of a portion of the accessory duct (see FIG. 1 and accompanying discussion below). For example, “a length of the pancreatic duct” refers to a length of the accessory duct alone, a length of the main pancreatic duct alone, a length that represents the combined lengths of the accessory duct and the main pancreatic duct, a length that represents the combination of less than a full length of the accessory duct and a full length of the main duct, a length that represents the combination of less than a full length of the main duct and a full length of the accessory duct, or a length that represents the combination of less than a full length of the main duct and less than a full length of the accessory duct.

As used herein with respect to the pancreatic duct, the terms “obstruct” and “occlude” include: (a) any act that reduces the volume of free space in the pancreatic duct lumen as compared to immediately before the action, and/or (b) any act that breaks, impairs, interrupts, slows, and/or renders impossible the flow of fluids (i) into the pancreatic duct lumen (e.g., from the exocrine ducts), (ii) through at least a portion of the pancreatic duct lumen, and/or (iii) through or within the exocrine ducts. It should be appreciated then that “obstruct” and “occlude,” as used herein, include any type of ductal ligation. Likewise, the terms “obstruction” and “occlusion” refer to any device(s) and/or substance(s) that achieve the action of “obstructing” or “occluding.”

As used herein with respect to the pancreas, “distal” refers to a position farther from the clinician along the longitudinal axis of the delivery system and “proximal” refers to a position closer to the clinician along the longitudinal axis of the delivery system.

As used herein with respect to biological tissue, the term “modulate” includes altering the physical structure of the tissue and/or rendering one or more cells of the tissue inert, inactive, or otherwise completely or partially reduced in function. Tissue modulation can be mechanically-induced (e.g., placing a device in the pancreatic duct lumen to obstruct the pancreatic duct lumen), electrically-induced, thermally-induced, or chemically-induced.

### II. Overview of Pancreatic Anatomy and Physiology

FIG. 1 is a schematic illustration of a human pancreas P and a portion of the adjacent duodenum D. The pancreas P is shown in cross-section, and a portion of the duodenal wall has been removed for ease of viewing the passages between the pancreas P and the duodenum D. The pancreas is both: (a) a part of the gastrointestinal system in that it that makes and secretes digestive enzymes into the intestine, and (b) an endocrine organ in that it makes and secretes hormones into the blood to control energy metabolism and storage throughout the body. The pancreas lies in the upper abdomen behind the stomach within the retroperitoneal space (i.e., in the abdominal cavity behind the peritoneum).

The pancreas is surrounded by a fibrous capsule from which connective tissue septa extend into the gland dividing its parenchyma into distinct lobes and lobules. For descriptive purposes, the pancreas is usually divided into the head, the body and the tail, although there are no clear-cut macroscopic borders between these major parts. Generally, the left border of the superior mesenteric vein SM (see FIG. 16B) is regarded as the border between the head aligned with the upper duodenum on the right and the body located underneath the stomach and extending roughly horizontally in the medial plane on the left. The mid-point of the body and tail combined is then arbitrarily defined as the border between the body and the tail, with the tail usually ranging from about 1.5-3.5 cm in length. Some authors define a fourth and a fifth part, the inferomedial uncinate process that lies beneath the SM and the superior mesenteric artery SM (see FIG. 16A), and the isthmus or neck, which is an approximately 2 cm wide part of the pancreas situated anterior to the SM and the point where the SMV and the splenic vein S (see FIG. 16B) join to form the portal vein.

The main pancreatic duct PD (or “duct of Wirsung”) has a mean diameter of 1.3 mm with the largest diameter at the head of the pancreas and a progressively smaller diameter on reaching the tail. The pancreas also has a secondary accessory duct AD known as “the duct of Santorini” or “the accessory pancreatic duct.” The main pancreatic duct joins with the bile duct BD to empty into the duodenum. The length of the main pancreatic duct PD is between 9.5 cm to 25 cm. The main pancreatic duct PD empties directly into the duodenum D through a perforation known as the major duodenal papilla MAPD, and the accessory pancreatic duct empties into the duodenum at a perforation called the minor duodenal papilla (MIPD).

The pancreas is about 14-18 cm long (measured from the head to the tail), about 2-9 cm wide, about 2-3 cm thick.

The pancreas comprises exocrine tissue EX and endocrine tissue EN. The exocrine tissue EX of the pancreas produces and secretes digestive enzymes into the duodenum D (e.g., trypsin, chymotrypsin, bicarbonate ions, lipase, amylase, phospholipase, etc.) to break down digestible foods within the digestive tract. Exocrine tissue comprises groups of acinar and duct cells (only two depicted in FIG. 1 for ease of viewing the rest of the pancreatic anatomy). The acinar cells are associated connective tissue, vessels, and nerves. Pancreatic juices (containing the digestive enzymes) are first secreted into a lumen of each acinus. The juices accumulate within these ducts and eventually drain into the main pancreatic duct PD. The exocrine components comprise more than 95% of the pancreatic mass.

The endocrine tissue EN of the pancreas produces and secretes hormones such as insulin, glucagon, somatostatin, and others into the bloodstream. The endocrine tissue is comprised of islets EN (or “islets of Langerhans”) and accounts for only 1-2% of the pancreatic mass.

The anatomy and physiology of the pancreas present several challenges when attempting to modulate pancreatic tissue. For instance, the deep and central location of the pancreas in the abdomen, coupled with its “wet sponge” texture, make it difficult to manipulate and/or grab hold of. Adding to this complexity, the pancreas lacks a capsule, or covering, and thus is prone to bleed or leak acidic juices with even a small degree of rough handling.

### III. Selected Intraductal Approaches to Modulating the Pancreas

FIG. 2 illustrates a system 100 for modulating retroperitoneal tissue, such as pancreatic tissue, according to one or more embodiments of the present technology. As shown in FIG. 2, the treatment system 100 has a proximal portion 100a configured to be extracorporeally positioned during treatment and a distal portion 100b configured to be endoluminally positioned at a treatment site at, within, or otherwise proximate to the pancreatic duct. The treatment system 100 may include a handle 102 at the proximal portion 100a and an elongated shaft 104 extending between the handle 102 and the distal portion 100b. The elongated shaft 104 may comprise a generally tubular structure surrounding one or more channels or lumens extending therethrough, and the treatment system 100 may comprise one or more treatment devices 200 configured to be delivered to the treatment site through the one or more lumens of the shaft 104. The elongated shaft 104 and/or the treatment device 200 may be configured to track over a guidewire 106 to the treatment site. In some embodiments, the treatment system 100 comprises only the treatment device 200 and does not include a separate elongated shaft through which the treatment device 200 is delivered (i.e., the treatment device 200 includes its own delivery shaft or otherwise does not require a delivery shaft).

The treatment device 200 may comprise an elongated member 204 and a treatment element 202 (shown schematically in FIG. 2) carried by a distal portion of the elongated member 204. As described in greater detail below, the treatment device 200 may be configured to position and deploy the treatment element 202 along a portion of the pancreatic duct to partially or fully obstruct the pancreatic duct lumen, thereby inducing atrophy of pancreatic cells (such as exocrine tissue). Without being bound by theory, it is believed that obstructing the flow of exocrine secretions into and/or out of the pancreatic duct causes necrosis of the pancreatic tissue. As such, the treatment devices of the present technology are configured to permanently functionally disable the diseased pancreatic tissue without the cost, time, and risk of surgery, thereby providing therapeutic relief to the patient.

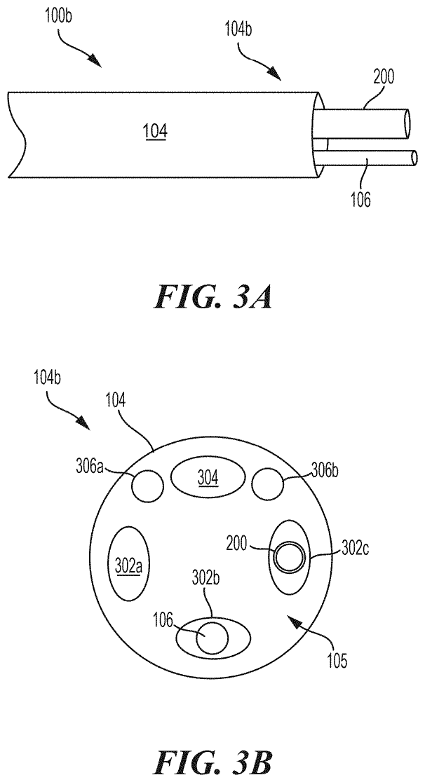

FIG. 3A is an enlarged side view of a distal portion 100b of a treatment system in accordance with some embodiments of the present technology, and FIG. 3B is an end view of the elongated shaft 104. The elongated shaft 104 is shown in FIGS. 3A and 3B with a treatment device 200 and a guidewire 106 positioned partially within two of its lumens. As shown, the elongated shaft 104 may comprise a generally tubular structure that contains a plurality of channels or lumens that extend from the proximal portion 100a of the treatment system 100 to a port or opening at the distal end 104b of the elongated shaft 104. In some embodiments, some or all of the channels may be discrete catheters or elongated tubular members that run together through a main lumen of the elongated shaft 104. In some embodiments the elongated shaft 104 may be generally solid in cross-section and the channels are cut through the solid cross-section. In some embodiments, the elongated shaft 104 comprises an endoscope.

The elongated shaft 104 may be configured to carry or slidably receive within its channels one or more interventional elements (such as the treatment element 202 of the present technology), as well as one or more accessory devices to aid in guiding the shaft 104 to a target treatment site and provide visualization of the procedure at the site. In the example shown in FIG. 3B, the elongated shaft 104 includes (a) an optical channel with a transducer extending therethrough and coupled at its distal end to a camera 304 (e.g., for enabling a video feed to the user), (b) multiple irrigation channels extending therethrough, each terminating distally at corresponding irrigation ports 306a and 306b, and (c) three separate and distinct tool channels extending therethrough and terminating distally at corresponding tool ports 302a-302c. The handle 102 (FIG. 2) may include one or more controls 103 (only a few labeled in FIG. 2) for manipulating various system components at the distal end of the shaft 104. For example, the controls 103 may be configured to turn the video feed on/off, mechanically adjust and move the distal portion of the shaft 104, deliver a fluid (such as contrast), advance/withdraw and/or rotate any portion of the treatment device 200, deploy the treatment element 202, and others.

As shown in FIG. 3B, in some embodiments the irrigation ports 306a, 306b may be positioned on either side of the optical port 304. In other embodiments, the irrigation ports 306a, 306b may be positioned at other locations along the distal face 105 of the elongated shaft 104 and/or may be arranged in other configurations with respect to the other ports. The tool ports 302a-302c may be spaced apart circumferentially around a periphery of the distal face 105 or may have other arrangements. The tool ports 302a-302c may be configured to slidably receive a guidewire and/or corresponding treatment device 200 therethrough. Although FIGS. 3A-3B show a treatment device 200 extending from tool port 302c and a guidewire 106 extending from tool port 302b, any of the tool ports 302a-302c may receive a treatment device 200 or guidewire 106 therethrough. Moreover, in some embodiments the guidewire 106 and treatment device 200 may be delivered through the same tool channel, for example with the treatment device 200 being advanced over the guidewire 106. In addition, one or more of the tool channels and corresponding ports 302a-302c may be configured to deliver a treatment substance (such as a glue, a liquid embolic, or other occlusive material) therethrough.

Although six channels and corresponding ports are depicted in the examples shown in FIGS. 3A and 3B, in other embodiments the elongated shaft 104 may have more or fewer channels and/or ports. For example, in some embodiments the elongated shaft 104 may include only a single channel or lumen extending therethrough. In such embodiments, the treatment device 200 may be configured to be slidably received through the shaft 104 lumen. In particular embodiments, the elongated shaft 104 may include two, three, four, five, seven, eight, nine, ten, or more channels and/or ports. The elongated shaft 104 may include more or fewer than three tool channels/ports (e.g., one tool channel/port, two tool channel/ports, four tool channel/ports, etc.), more or fewer than two irrigation channels/ports (e.g., one irrigation channel/port, three irrigation channels/ports, etc., and/or more than a single optical channels/ports (e.g., two optical channels/ports, three optical channels/ports, etc.). The elongated shaft 104 may also comprise different spatial arrangements of the channels and/or ports than that shown in FIGS. 3A and 3B.

Moreover, the elongated shaft 104 may include different combinations of channels/ports than that shown in FIGS. 3A and 3B. In some embodiments of the technology, the elongated shaft 104 may comprise a tool channel(s)/port(s) and an irrigation channel(s)/port(s) but does not include an optical port. In some embodiments the elongated shaft 104 may comprise a tool channel(s)/port(s) and an optical channel(s)/port(s) but does not include an irrigation port. In some embodiments of the technology, the elongated shaft 104 may comprise a tool channel(s)/port(s) in combination with a type of channel(s)/port(s) other than an optical channel and an irrigation channel. For example, in some embodiments the treatment system 100 may include an intravascular ultrasound probe, and the elongated shaft 104 may have a channel to receive the probe therethrough.

In some embodiments the elongated shaft 104 is configured to remain outside of the pancreatic duct (usually in the duodenum) while the treatment device 200 is advanced from a distal port of the shaft 104 and used to navigate the pancreatic duct. Additionally or alternatively, the elongated shaft 104 itself may be configured to navigate all or a portion of the pancreatic duct. In any case, the diameter of the elongated shaft 104 at its distal portion 104b may be sized to navigate the main pancreatic duct and/or the accessory duct. Traditional devices for accessing the proximal portion of the endoscopic retrograde cholangiopancreatography (“ERCP”) scope. Traditional ERCP scopes, for example, have a distal diameter of about 7-12 mm, which is too large to traverse the much smaller diameter (1-3 mm) of the pancreatic duct. The elongated shaft 104 may therefore have a diameter sized to navigate the main pancreatic duct and/or the accessory duct. The elongated shaft 104 may have a diameter that is less than or equal to 7 mm, 6 mm, 5 mm, 4 mm, 2 mm, or 1 mm. Preferably, the distal diameter of the elongated shaft 104 is less than the diameter of the pancreatic duct, which is typically about 3 mm.

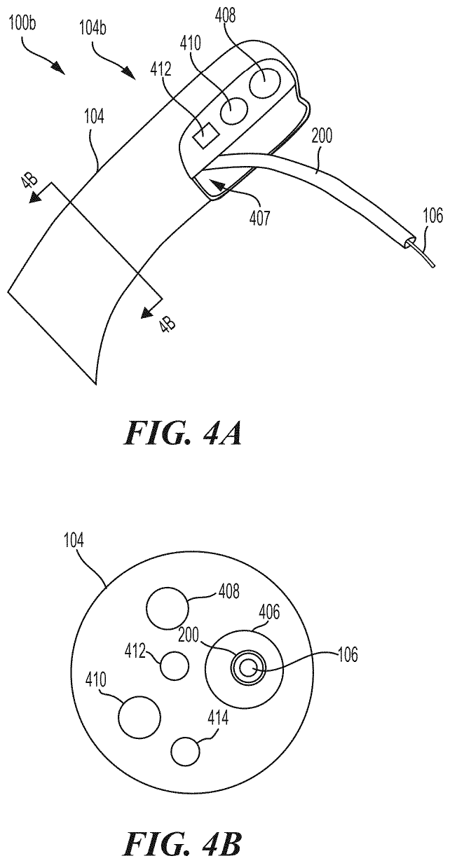

In the example elongated shaft 104 shown in FIGS. 3A and 3B, the exit ports are generally perpendicular to the longitudinal axis of the shaft 104 such that the treatment device 200 initially exits the shaft 104 generally parallel with the longitudinal axis of the shaft 104. In some embodiments, however, it may be beneficial to have the distally extending components of the system exit the elongated shaft 104 through a side-facing exit port to help navigate sharp turns along the delivery path. FIG. 4A, for example, shows the distal portion 100b of a treatment system 100 in which the elongated shaft 104 comprises an elongated tubular member having a laterally-facing opening 407 in its sidewall at its distal end portion 104b. Similar to the elongated shaft 104 shown in FIGS. 3A and 3B, the elongated shaft 104 of FIGS. 4A and 4B may include a plurality of channels or lumens extending therethrough that terminate at ports at the distal end of the shaft 104. The elongated shaft 104 may include, for example, an air/water nozzle 412, a water jet 414, a camera 408, a light 410, and a tool channel 406. The treatment device 200 may be slidably disposed within the channel 406 of the elongated shaft 104 and configured to extend through the side facing opening 407 and laterally away from the distal portion of the shaft 104b. In some embodiments, the elongated shaft 104 may include a shelf or ramp (not shown) proximate the opening 407 at the distal end of the channel 406 that deflects the treatment device 200 laterally away from the elongated shaft 104 as it exits through opening 407. In FIGS. 4A and 4B, the treatment device 200 is shown being advanced over a guidewire 106.

In some aspects of the technology, a steering wire can be attached to the distal portion of the elongated shaft 104 (such as either of those shown in FIGS. 3A-4B), such that creating tension in the steering steers the elongated shaft 104 and/or the elongated member of the treatment device into the pancreatic duct.

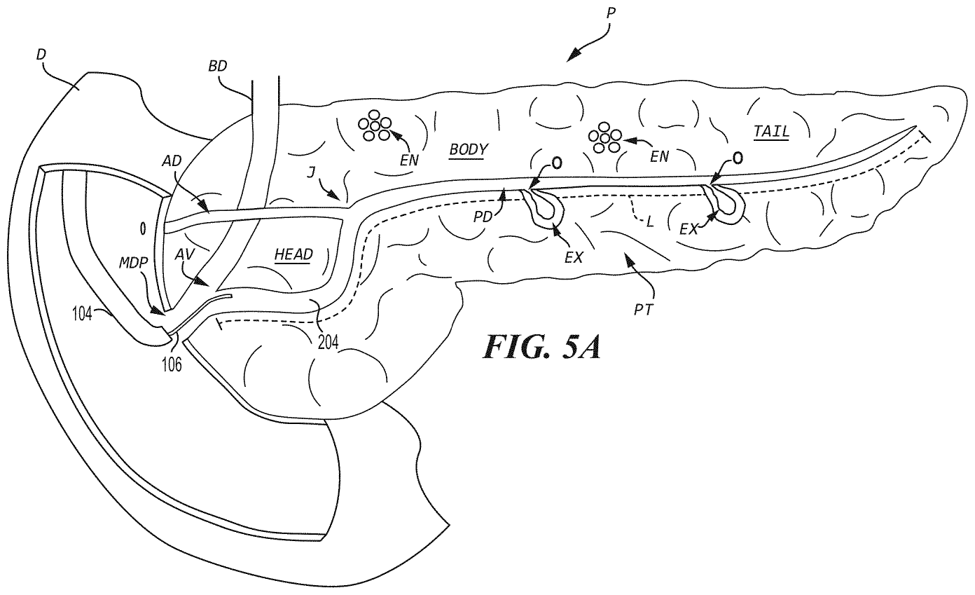

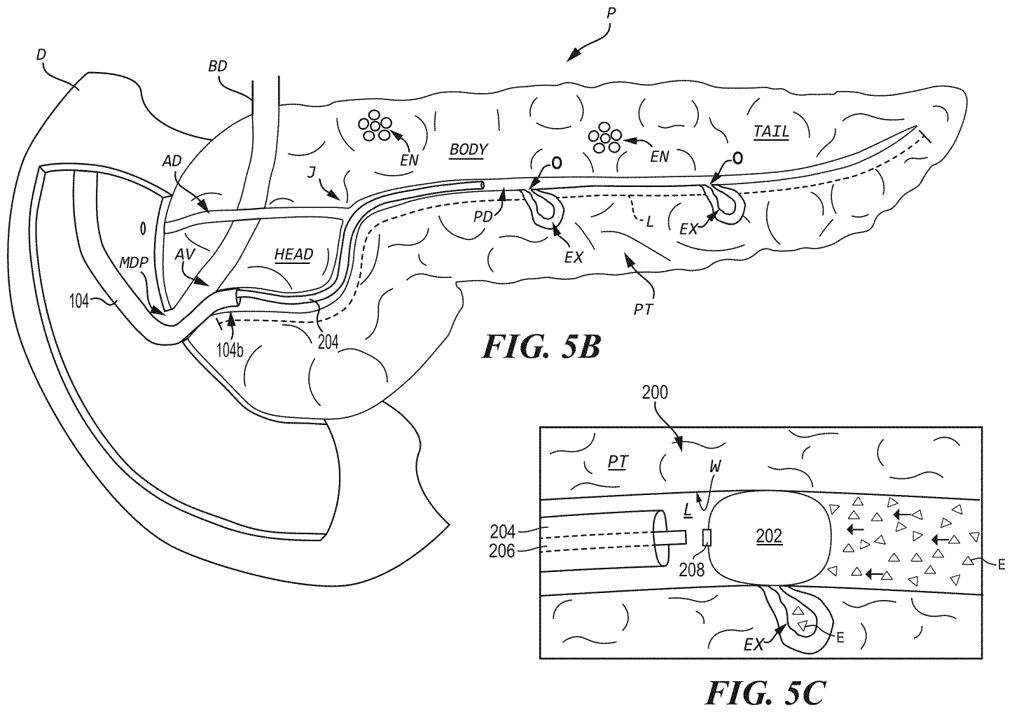

FIGS. 5A-5C illustrate an example method for obstructing a pancreatic duct lumen using the treatment systems 100 of the present technology (such as the treatment systems shown in FIGS. 3A-4B). In use, the distal portion 104b of the elongated shaft 104 may be inserted through the mouth of the patient and advanced down the esophagus, through the stomach, through the pylorus, and through the duodenum D until the distal portion 104b is proximate the major duodenal papilla MPD. At any time during the procedure, a catheter (not shown) may be delivered through one of the tool channels and advanced through the major duodenal papilla MPD to the ampulla of Vater AV to deliver radiocontrast into the bile duct BD and/or pancreatic duct PD for better visualization during the procedure. Additionally or alternatively, the elongated shaft 104 may have a built-in fluid lumen terminating at a fluid outlet at the distal end of the shaft 104, and through which radiocontrast may be delivered.

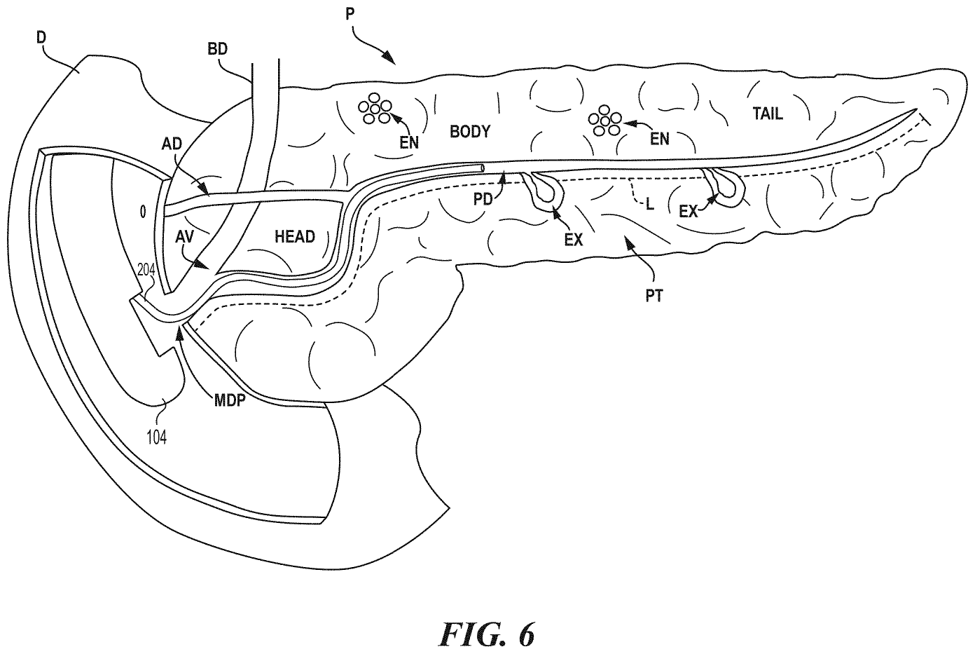

As shown in FIG. 5A, with the distal portion 104b of the elongated shaft 104 within the duodenum D proximate the MPD, a guidewire 106 (see FIG. 2) may be advanced through the opening at the distal end of the elongated shaft 104, through the MPD, and past the ampulla of Vater AV to a proximal portion of the pancreatic duct PD. As shown in FIG. 5B, the elongated shaft 104 may then be advanced over the guidewire 106 into at least the proximal portion of the pancreatic duct PD, and the guidewire 106 may be withdrawn. In some embodiments, the elongated shaft 104 remains within the duodenum D while the treatment device 200 is advanced distally over the guidewire 106 into the pancreatic duct PD. The elongated shaft 104 shown in FIGS. 4A and 4B may also be used in this manner, as depicted in FIG. 6. While the ensuing description of the system 100 refers to deploying the treatment element 202 through an elongated tubular member 204 of the treatment device 200 that has been advanced through a lumen of the elongated shaft 104, it will be appreciated that this description also applies to embodiments in which the treatment element 202 is advanced through and deployed directly from a lumen of the elongated shaft 104 without the use of a separate elongated tubular member 204.

The distal portion of the elongated member 204 of the treatment device 200 may be advanced to a location along the length of the pancreatic duct PD that is desirable for creating an obstruction (also referred to as a “treatment location”). As shown in the enlarged, isolated view of FIG. 5C, deployment of the treatment element 202 may cause the treatment element 202 to expand into apposition with an inner surface of the pancreatic duct wall W such that the treatment element 202 obstructs at least a portion of the cross-sectional area of the pancreatic duct lumen L. In some embodiments, deploying the treatment element 202 includes expanding the treatment element 202 into apposition with an inner surface of the pancreatic duct wall W such that the treatment element 202 obstructs substantially the entire cross-sectional area of the pancreatic duct lumen L. In any case, deployment of the treatment element 202 within the pancreatic duct lumen L blocks a sufficient cross-sectional area of the lumen L such that exocrine duct EX secretions E are substantially prevented from travelling along the pancreatic duct PD proximal of the treatment element 202 (sometimes referred to herein as an “intraluminal obstruction”). Additionally or alternatively, deployment of the treatment element 202 may cover or otherwise render impassable one or more exocrine duct EX openings O (see FIG. 5B) along the pancreatic duct PD through which the secretions E normally enter the pancreatic duct lumen L (sometimes referred to herein as an “interluminal obstruction”). The presence of these interluminal and/or intraluminal obstructions within the pancreatic duct PD causes an accumulation of digestive enzymes within the pancreatic tissue PT, thereby inducing atrophy and eventually necrosis of pancreas cells. Depending on the placement and/or extent of the obstruction(s), the treatment element 202 may cause partial or complete necrosis of pancreatic tissue.

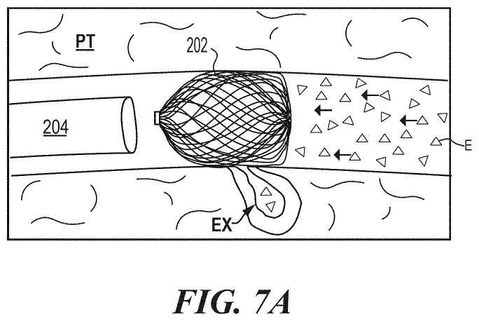

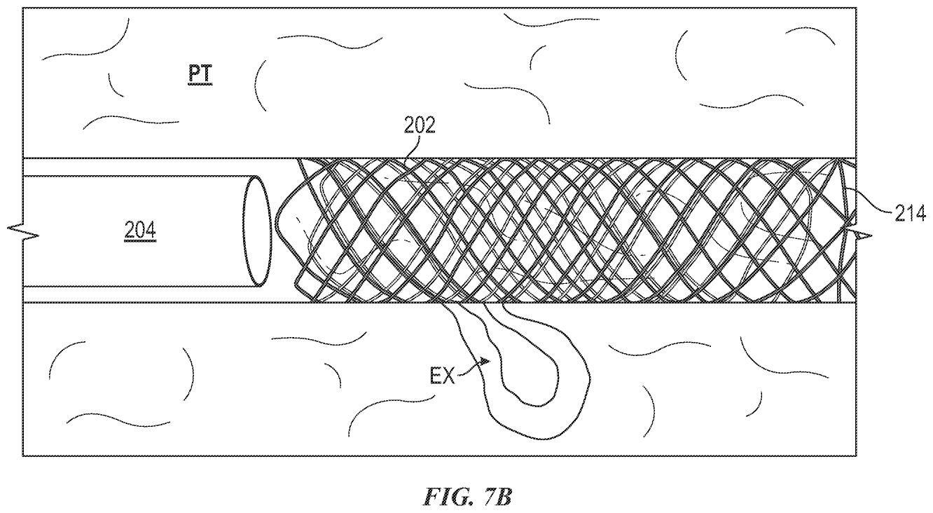

As depicted schematically in FIG. 5C, in some aspects of the technology, the treatment element 202 may be an expandable device having a collapsed or constrained configuration for delivery through the elongated shaft 104 and/or elongated member 204 and an expanded configuration for implantation within the duct lumen L. The treatment element 202 may be self-expanding such that once released from the lumen of the elongated member 204, the treatment element 202 self-expands into apposition with an inner surface of the duct wall W. As shown in FIG. 5C, the treatment device 200 may include a first elongated member 204 in the form of an elongated tubular shaft and a second elongated member 204 having a distal end configured to detachably couple to a coupler 208 of the treatment element 202. The treatment element 202 may be configured to be delivered through the first elongated member 204 in a collapsed or constrained configuration. Once reaching the treatment site, the first elongated member 204 may be moved proximally relative to the second elongated member 206 (e.g., by pulling the first elongated member 204 proximally while hold the second elongated member 206 in place or by pushing the second elongated member 206 while hold the first elongated member 204 in place) thereby releasing the treatment element 202 from the constraints of the first elongated member 204 and allowing the treatment element 202 to expand into contact with the duct wall W. The clinician may then detach the treatment element 202 from the second elongated member 206 and withdraw the first and second elongated members 204 and 206, thereby leaving the treatment element 202 implanted in the pancreatic duct lumen L. Additional treatment element details are discussed below with reference to FIGS. 7A-10.

The obstructions may partially or totally occlude the pancreatic duct lumen L along all or a portion of the length of the pancreatic duct PD. In some cases it may be beneficial to obstruct greater portions of the pancreatic duct, as doing so may accelerate tissue necrosis. Without being bound by theory, it is believed that the rate of acinar cell destruction is mediated by the extent of obstruction to the main pancreatic duct. More complete ductal obstruction would thereby be associated with a higher concentration of digestive enzymes accumulating in the acinar glands and accelerating destruction of the tissue. This would potentially affect the time it takes for the pancreatic exocrine tissue to necrose, by shortening the time needed to reach the full therapeutic effect of treatment. As previously mentioned, the treatment system 100 may include an ultrasound probe to measure intraductal pressure while initiating ligation. This would help systematize and make uniform the process of ligation for reproducibility.

In some cases it may be beneficial to obstruct the duct lumen at multiple locations via placement of multiple treatment elements 202. For example, in some embodiments the method may include deploying a first treatment element 202 (such as any of the treatment elements disclosed herein) at a first location along the pancreatic duct PD and deploying a second treatment element 202 (such as any of the treatment elements 202 disclosed here) at a second location along the pancreatic duct. The first and second treatment elements 202 may have the same or different physical structure (examples of which are discussed in greater detail below), the same or different length, the same or different maximum cross-sectional area, the same or different minimum cross-sectional area, and/or may be configured to cause the same or different degree of occlusion within the lumen.

The obstructions may extend along or affect all or a portion of the pancreatic duct PD. In some embodiments, the method may include deploying a first treatment element 202 at a first location within the lumen of the main pancreatic duct PD and deploying a second treatment element 202 at a second location within the lumen of the accessory pancreatic duct AD. In some embodiments, a single treatment element may completely or partially obstruct at least a portion of the main pancreatic duct PD and completely or partially obstruct at least a portion of the accessory pancreatic duct AD. In some embodiments, a single treatment element may completely or partially obstruct substantially the entire length of the main pancreatic duct PD and may completely or partially obstruct substantially the entire length of the accessory pancreatic duct AD.

In various embodiments of the technology, the treatment element 202 may be configured to contact substantially the full circumference of the inner surface of the duct wall along substantially the entire length of the treatment element 202. For example, in some embodiments the treatment element 202 may be substantially cylindrical in its expanded state with an unconstrained diameter that is slightly greater than that of the targeted region of the pancreatic duct PD. As a result, when the substantially cylindrical treatment element 202 is positioned within the duct, the treatment element 202 contacts and presses outwardly against substantially the entire circumference of the duct wall along substantially the entire length of the treatment element 202. This contact and outward force helps to secure the treatment element 202 within the pancreatic duct PD as well as seal off any exocrine duct openings O (FIG. 5B) adjacent the treatment element 202 along the duct PD. The positioning of the treatment element 202 over the openings O substantially prevents the exocrine secretions E (FIG. 5C) within the ducts from reaching the pancreatic duct lumen.

In some embodiments, the treatment element 202 may be configured to contact substantially the full circumference of the inner surface of the duct wall along less than the entire length of the treatment element 202. For example, in those embodiments where at least a portion of the treatment element 202 has a curved outer surface (e.g., spherical, ovoid, egg-shaped, etc.), the treatment element 202 may contact a full circumference of the inner surface of the duct wall only along an intermediate portion of the treatment element's length and not at its proximal and distal-most regions.

### A. Selected Embodiments of Occlusive Devices

As shown in FIG. 5C, in some embodiments the treatment element 202 may comprise an occlusive, expandable element. The expandable element, for example, may be formed of a hollow metal structure comprising a continuous metal wall that defines a cavity therein. The expandable element may have a compressed state for delivery through the elongated shaft 104 and/or the elongated member 204, and an expanded state in which the metal wall is radially expanded. The wall may have an opening therethrough that allows for the passage of fluid into the cavity to inflate or otherwise expand the metal structure. For example, the treatment device 200 may include an elongated member (such as the second elongated member 206) coupled at its proximal end to a fluid source (not shown) and detachably coupled at its distal end to the metal structure. The elongated shaft may include a fluid lumen that is in fluid communication with the interior cavity. The compressed hollow structure may be configured to expand in response to receiving fluid into its cavity.

The expanded shape of the metal structure may be selected based on the desired amount of occlusion. As such, the preferred shape is round, or generally rounded. Also, the expanded treatment element comprises a single lobe to maximize the wall contact between the expanded treatment element and the luminal surface of the pancreatic duct wall so as to reduce the risk of the treatment element migrating within or out of the pancreatic duct, and to increase the coverage of the exocrine duct EX openings along the duct wall. The wall of the metal structure can be uniform or variable, with the thickness changing at different locations on the metal structure. In some embodiments, the wall of the region near the coupler 208 is thicker than the main body of the metal structure, while in other embodiments this region is thinner.

In some embodiments, an external surface of the wall of the metal structure includes projections configured to reduce migration after expansion. These projections may be macroscopic, such as with the hooks or barbs seen on other implanted cardiovascular medical devices such as caval filters. For example, a plurality of projections, such as barbs and hooks, can be located on the exterior layer to anchor the treatment element to the surrounding tissue. In a further embodiment, these projections comprise an expansile metal, such as nitinol. For some embodiments, these projections are microscopic, ranging in length from 0.01 pm to about 57 pm. In other embodiments, these projections are branching and can be made of nitinol or fibers.

The surface of the treatment element wall can be configured to increase local tissue growth into the metal wall in order to secure the treatment element in place and reduce the risk of treatment element migration. The wall of the treatment element can further be configured to release solutions that can include drugs, pharmacologically active molecules, or pharmacologic compositions, such as those that would expedite necrosis of pancreatic tissue by blocking one or more exocrine duct openings and/or drugs that stimulate cell proliferation or the production of extracellular matrix, or increase the rate or extent of tissue growth, such as tissue growth into pores, or around projections, of the wall of the treatment element.

In some embodiments, the treatment element 202 may comprise an expandable mesh. Examples of expandable meshes of the present technology are depicted in FIGS. 7A and 7B. The mesh elements of the present technology have a low-profile or constrained state while positioned within the elongated shaft 104 and/or the elongated member 204 for delivery to a deployment location within the pancreatic duct and an expanded state in which at least a portion of the mesh is configured to be in apposition with all or a portion of the circumference of the luminal wall at the treatment site. In some embodiments, the expandable mesh may have a sufficiently low porosity so that the mesh substantially prevents egress of the exocrine secretions.

In some embodiments, the mesh can be made of a plurality of filaments or struts. In some embodiments, the mesh can be braided, woven, molded, or cut from a sheet or tube. The filaments or struts can be formed of known flexible materials including shape memory materials (e.g., nitinol), cobalt chromium, platinum, stainless steel, other metals, other metal alloys, or a combination thereof. In some embodiments, all or some of the filaments or struts may be formed of one or more biodegradable metals, such as a metal comprising iron, magnesium, and/or zinc. In some embodiments, the filaments can be wire having a round, ovoid, square, rectangular, or other shape in cross-section. Further, the filaments or struts can be configured such that the treatment element 202 is self-expanding. In some embodiments, at least a portion of the treatment element 202 will tend to resiliently assume an expanded configuration in the absence of a countervailing force. The filaments may comprise metal and/or polymer wires. One, some, or all of the filaments forming the mesh may be formed of a drawn-filled tube wire comprising a core material surrounded by an outer material. The core material may be a radiopaque material, such as platinum, and the outer material may be a shape memory alloy, such as nitinol, chromium cobalt (“CrCo”) alloys, stainless steel alloys, etc.

The wire filaments can be braided into a resulting lattice-like structure. In at least one embodiment, during braiding or winding of the treatment element 202, the filaments can be braided using a 1-over-2-under-2 pattern. In other embodiments, however, other methods of braiding can be followed, without departing from the scope of the disclosure. Such other braiding methods can include a 1-over-1-under-1 pattern and 2-over-2-under-2 pattern. In some embodiments, the treatment element 202 be heat set to a desired shape, such as, for example, by placing the treatment element 202 in contact with a molding surface of a molding element which defines a desired shape of all or a portion of the treatment element 202.

The treatment element 202 can comprise pores. In some embodiments, the treatment element 202 can exhibit a porosity configured to reduce or prevent egress of exocrine secretions. For example, if the treatment element 202 is formed of a braid, the sizes of the pores can be controlled by adjusting the numbers of wires in the braid and the pick and pitch of the braid. As will be appreciated, the porosity of the treatment element 202 can be adjusted by longitudinally “packing” the treatment element 202 during deployment, as known in the art. In some embodiments, all or a portion of the mesh may be covered by a cover, such as a polymer liner or Dacron material. The cover may be positioned over a distal end opening of the mesh (for example as depicted by cover 214 in FIG. 7B), a proximal end of the mesh, and/or the sidewalls of the mesh.

In some embodiments, the treatment element 202, whether or not it comprises a plurality of filaments, can be coated or surface-treated with one or more compounds, such as, for example, antithrombotic agents.

The treatment element 202 may be formed of 24, 32, 36, 48, 64, 72, 96, 128, or 144 filaments. The treatment element 202 may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments 144 of the braided mesh 142 may have the same diameter. For example, in some embodiments, all of the filaments 144 have a diameter of about 0.001 inches. In some embodiments, some of the filaments 144 may have different cross-sectional diameters. For example, some of the filaments 144 may have a slightly thicker diameter to impart additional strength to the braided layers. In some embodiments, some of the filaments can have a diameter of about 0.001 inches, and some of the filaments can have a diameter of greater than 0.001 inches. The thicker filaments may impart greater strength to the braid without significantly increasing the device delivery profile, with the thinner wires offering some strength while filling-out the braid matrix density.

While the treatment element 202 is self-expanding and thus easy to deploy, the fine braided filaments forming the sidewalls of the treatment element make it sufficiently soft and compressible to avoid injuring delicate tissue (such as the wall of the pancreatic duct). The treatment element 202 can therefore be sufficiently self-expanding to effectively move or capture thrombus, without posing a risk of injuring the surrounding vessel.

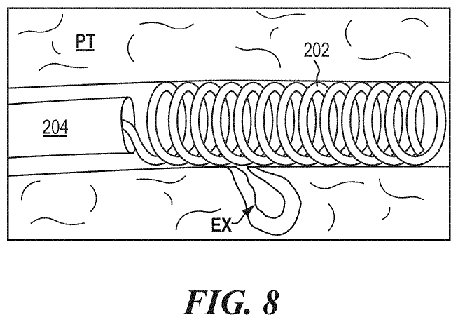

FIG. 8 depicts an example of a treatment element 202 comprising a coil. The coil may be delivered to the ductal lumen L such that an outer surface of the coil is in apposition with an inner surface of the duct wall. In some embodiments, the coil may include one or more fibers. After insertion, scar tissue forms around the coils, thereby forming intraluminal and/or interluminal obstructions. After the procedure, scar tissue forms around the coils, thereby partially or completely intra- and/or inter-luminally obstructing the pancreatic duct to prevent or substantially prevent exocrine duct secretions from reaching the pancreatic duct lumen and/or traveling proximally through the duct and exiting into the duodenum D via the MPD (FIG. 5B).

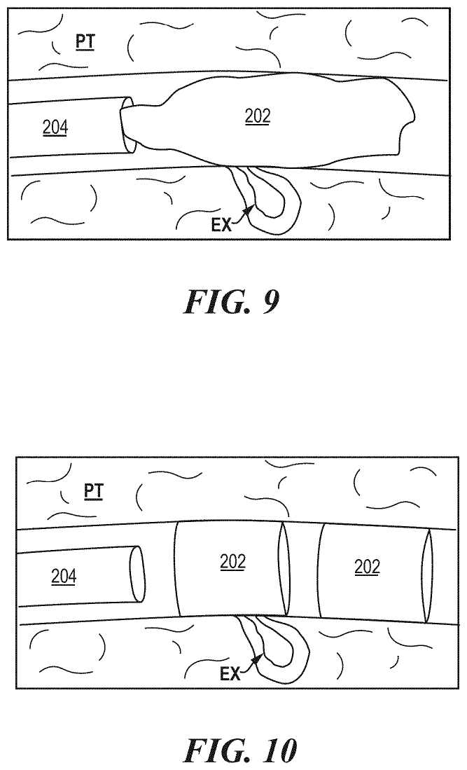

FIG. 9 shows an example of a treatment device 200 of the present technology in which the treatment element 202 comprises a flowable (at least at the time of delivery) occlusive substance that can be injected through the elongated member 204 and/or the elongated shaft 104 into the pancreatic duct PD to partially or completely occlude or obstruct the pancreatic duct PD. The occlusive substance may comprise one or more of a glue, a foam, a hydrogel, a liquid embolic, and other suitable materials. The occlusive substance may be delivered to fill all or a portion of the length of the pancreatic duct PD. Along its length, the occlusive substance may partially or completely cover the cross-sectional area of the lumen. The occlusive substance is thus configured to intra- and/or inter-luminally obstruct the pancreatic duct to prevent or substantially prevent exocrine duct secretions from reaching the pancreatic duct lumen and/or traveling proximally through the duct and exiting into the duodenum D via the MPD (FIG. 5B).

FIG. 10 depicts another example of a treatment element 202 of the present technology configured to create an obstruction at or within the pancreatic duct lumen L. As shown in FIG. 10, the treatment element 202 may comprise one or more flexible occlusive members configured to be positioned in the pancreatic duct lumen L. In some embodiments, the members may be made of silicone, similar to those used to obstruct fallopian tubes. In practice, the clinician heats a small portion of the pancreatic duct via a heating element positioned within the pancreatic duct, at or around the outer circumference of the pancreatic duct, or both. The clinician may then insert one or more flexible occlusion members. After the procedure, scar tissue forms around the flexible inserts, thereby partially or completely intra- and/or interluminally obstructing the pancreatic duct to prevent or substantially prevent exocrine duct secretions from reaching the pancreatic duct lumen and/or traveling proximally through the duct and exiting into the duodenum D via the MPD (FIG. 5B). Although two occlusive members are shown in FIG. 10, the treatment devices of the present technology may include and position a single occlusive member or more than two occlusive members. Moreover, although the depicted occlusion members have a generally cylindrical shape, in other embodiments the occlusion members may have other suitable shapes.

In some embodiments, the occlusive member may comprise a solid, generally cylindrical member that is placed longitudinally directly into the duct. The member may comprise a polymer, a metal, or both. The metal may be a non-biodegradable metal, or may be formed of a biodegradable metal (such as a metal comprising iron, zinc, and/or magnesium). In some embodiments, such an occlusive member may be formed partially or entirely of biodegradable materials. In some embodiments, such an occlusive member may be formed partially or entirely of non-biodegradable materials.

In some embodiments, the treatment device 200 may include a device configured to partially or completely close the lumen (thereby creating an obstruction) by pulling or twisting the duct wall to bring the duct wall together.

In some instances, it may be beneficial to employ other methods for occluding the pancreatic duct instead of or in addition to implantation of an occlusive device within the ductal lumen. For example, in some embodiments one or more components of the treatment device 200 may include an electrode(s) (not shown) configured to deliver radiofrequency (RF) energy to the duct wall. The electrodes may be carried by the expandable treatment element 202, the elongated member 200, and/or other components of the treatment device 200. In use, the ablation electrode(s) may be positioned in apposition with an inner surface of the duct wall, and the RF energy may be delivered directly to the pancreatic duct wall to destroy the exposed tissue and form scar tissue. In some embodiments, after delivering energy at a first location along the length of the pancreatic duct, the treatment device 200 may be repositioned at a new location along the duct and the energy may be delivered again. This process may be repeated as necessary. Additionally or alternatively, one or more components of the treatment device 200 may be configured to ablate the duct tissue via cryoablation.

In some embodiments of the technology, the treatment systems may be configured to deliver one or more sclerosing agents to the pancreatic duct, such as quinacrine, methyl cyanoacrylate (“MCA”), silver nitrate, and others. The one or more sclerosing agents may be used alone or in combination with one or more of the other ligation devices and methods disclosed herein. For example, in some embodiments, the treatment system may include an occlusive device (such as any of the occlusive members disclosed herein) and one or more sclerosing agents. The occlusive device may be positioned at a location along the pancreatic duct, and the sclerosing agent may be delivered proximal or distal of the location. In some embodiments, the sclerosing agent may be delivered at substantially the same location as the occlusive device. The sclerosing agent may be incorporated with and/or delivered by the occlusive device, or the sclerosing agent may be delivered via a separate device.

### IV. Selected Embodiments of Laparoscopic Modulation of Pancreas Tissue

In addition to the devices, systems, and methods detailed above for obstructing the pancreatic duct via an intraluminal approach, the present technology includes devices, systems, and methods for laparoscopic obstruction of the pancreatic duct. For example, laparoscopic methods of the present technology include making small incisions in the wall of the abdomen and inserting a laparoscope into one of the incisions. The distal end of the laparoscope may have an ultrasound probe for guidance. One or more devices of the present technology may be inserted through the same or other incisions to perform laparoscopic obstruction of the pancreatic duct. Examples of these devices and methods are described below.

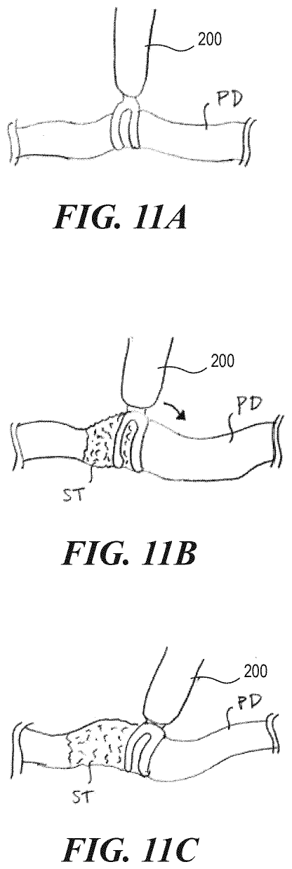

As shown in FIG. 11A, the treatment system 100 may include an electrocauterization device 200 configured to be delivered to an exterior portion of the pancreatic duct laparoscopically. As depicted in the progression of FIGS. 11A-11C, the electrocauterization device 200 may apply heat to the pancreatic duct wall to form scar tissue at the pancreatic wall. It is believed that this scar tissue creates an obstruction (interluminal and/or intraluminal) that slows or inhibits exocrine duct secretions into and/or through the pancreatic duct.

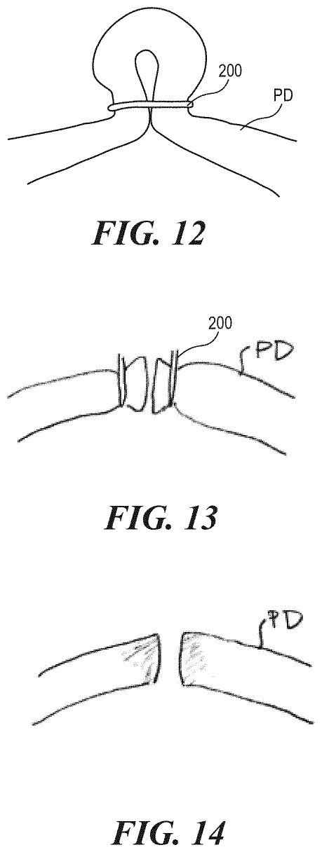

In some embodiments, the treatment systems of the present technology may be configured to create an obstruction at the pancreatic duct via other laparoscopic methods. For example, the treatment system may include a treatment device configured to laparoscopically cinch the duct together to create an obstruction (FIG. 12), cut and tie off the severed ends of the duct to create the obstruction (FIG. 13), and cauterize completely through the duct (FIG. 14).

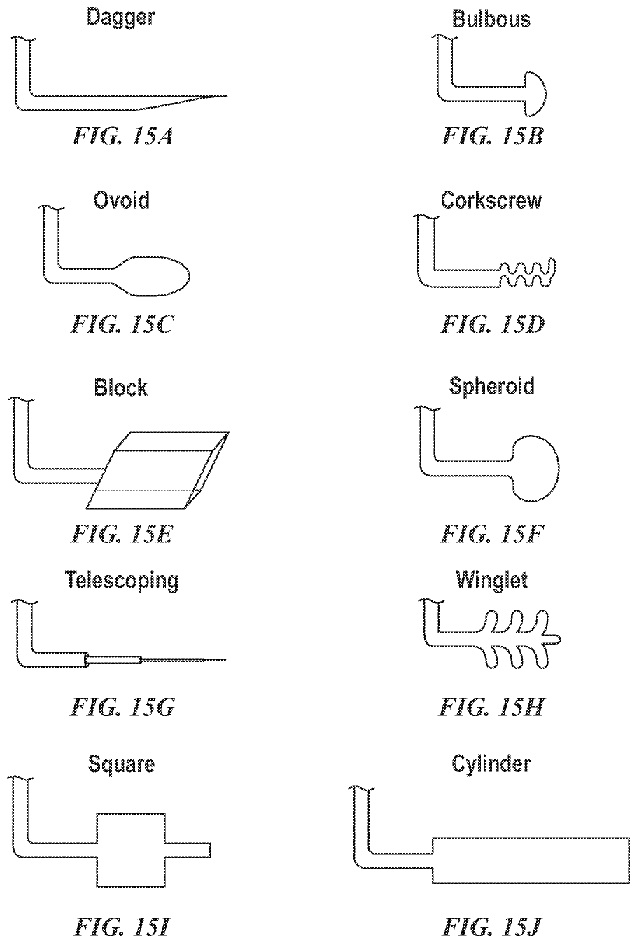

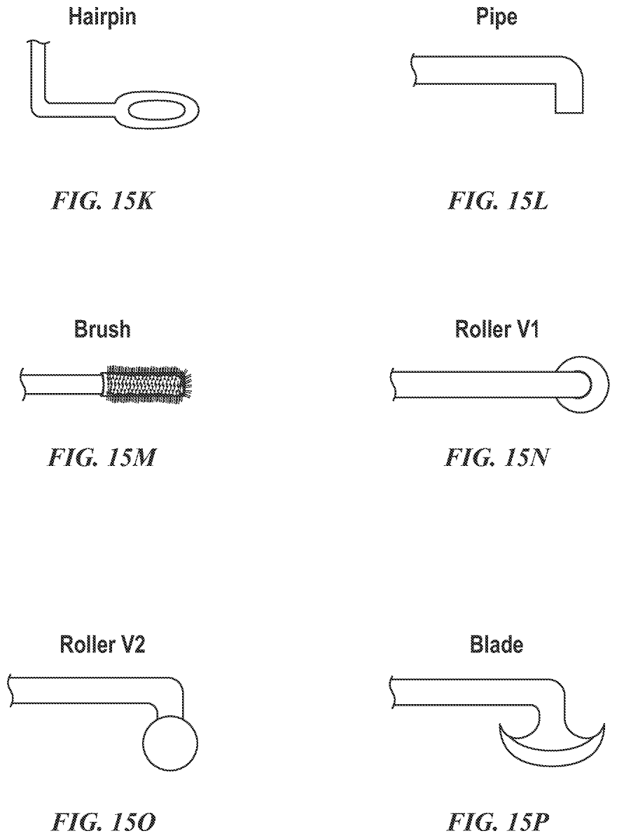

In some embodiments, the catheter may have a dagger shape (e.g., FIG. 15A), a bulbous shape (e.g., FIG. 15B), a corkscrew shape (e.g., FIG. 15D), a block shape (e.g., FIG. 15E), a spheroid shape (e.g., FIG. 15F), a telescoping shape (e.g., FIG. 15G), a winglet shape (e.g., FIG. 15H), a square shape (e.g., FIG. 15I), a cylindrical shape (e.g., FIG. 15J), a hairpin shape (e.g., FIG. 15K), a pipe shape (e.g., FIG. 15L), a brush shape (e.g., FIG. 15M), a first roller shape (e.g., FIG. 15N), a second roller shape (e.g., FIG. 15O), a blade shape (e.g., FIG. 15P).

### V. Selected Approaches to Vascular Modulation

The treatment devices and systems of the present technology may be used to modulate pancreatic tissue by obstructing or damaging one or more arteries or veins associated with the pancreas. Vascular modulation of pancreatic tissue may be used as a standalone therapy for treating conditions of the pancreas, or may be used in conjunction with any of the occlusive devices and methods discussed herein and/or any of the devices and methods for neuromodulating the nerves associated with the pancreas as described herein.

FIG. 16A depicts the major arteries feeding the pancreas. As shown in FIG. 16A, the blood supply of the pancreas is primarily derived from the celiac artery, representing approximately 1% of the total cardiac output. The head of the pancreas is supplied by an arterial arcade system formed by the anterior and posterior superior pancreaticoduodenal arteries SPD (only anterior labeled in FIG. 16A) arising from the gastroduodenal artery GD of the common hepatic artery CH and anastomosing with the anterior and posterior inferior pancreaticoduodenal artery IPD (only anterior labeled in FIG. 16A), which is the first branch of the superior mesenteric artery SM. The body and pancreatic tail primarily receive their blood supply from (a) the pancreatic branches of the splenic artery S, which is the branch of the celiac artery closest to the spleen, and (b) by the dorsal pancreatic artery DP that branches off near the origin of either celiac, hepatic, or splenic S artery. Its right branch anastomoses with the anterior superior pancreaticoduodenal artery SPD whereas its left branch forms the transverse pancreatic artery that runs along the inferior border of the pancreas and usually anastomoses with the pancreatica magna artery, the largest pancreatic branch of the splenic artery in the middle of the gland. A treatment device of the present technology may be configured to be positioned within a lumen of any of the aforementioned arteries to obstruct blood flow through the arteries, thereby modulating pancreatic tissue and treating one or more conditions of the pancreas, such as pancreatitis and pancreatic cancer.

Referring to FIG. 16B, the venous drainage of the pancreas mainly follows the arterial system and drains into the portal vein PV via either the splenic vein S, from the body and tail of the pancreas, or via the superior mesenteric vein SM from the pancreatic head venous arcade system. A treatment device of the present technology may be configured to be positioned within a lumen of any of the veins proximate the pancreas, such as the portal vein PV, the right gastric vein RG, the left gastric vein LG, the posterior superior pancreaticoduodenal vein PSPD, the anterior superior pancreaticoduodenal vein ASPD, the right gastroepiploic vein RGE, the left gastroepiploic vein LGE, the anterior inferior pancreaticoduodenal vein AIPD, the splenic vein S, the caudal pancreatic vein CP, the great pancreatic vein GP, the inferior mesenteric vein WI, the middle colic vein MC, and/or any branch or feeder vessel of one or more of the foregoing. When positioned in the targeted blood vessel, the treatment device may partially or completely obstruct blood flow through the blood vessel to modulate pancreatic tissue and treat one or more conditions of the pancreas, such as pancreatitis and pancreatic cancer.

### VI. Neuromodulation of Pancreatic Tissue

The treatment devices and systems of the present technology may be used to modulate pancreatic tissue via neuromodulation of one or more nerves innervating the pancreas. Neuromodulation of pancreatic tissue may be used as a standalone therapy for treating conditions of the pancreas, or may be used in conjunction with any of the occlusive devices and methods and/or vascular modulation devices and methods discussed herein.

As used herein, “neuromodulation” includes rendering neural fibers inert, inactive, or otherwise completely or partially reduced in function. This result can be electrically-induced, thermally-induced, or induced by another mechanism. (e.g., chemically-induced) during a neuromodulation procedure, e.g., a procedure including percutaneous transluminal intravascular access.

FIG. 17 is a dorsal view of a human pancreas P showing a partial innervation thereof. Many of the nerves that innervate the pancreas P extend from the distal end of the vagal trunk. The celiac trunk CT is one such extension that innervates the pancreas. Neural tissue known as the celiac plexus CP is located near where the celiac trunk CT, the superior mesenteric artery, and renal arteries branch from the abdominal aorta. Like other nerve plexuses, the celiac plexus CP includes a network of interconnecting nerve fibers and ganglia (e.g., celiac ganglia). The celiac ganglia can be detected, for example, using endoscopic ultrasound or other techniques (e.g., CT, fluoroscopy). For example, visualized ganglia are typically located adjacent to the celiac artery, anterior to the aorta, and are predominantly oval or almond-shaped, ranging in size from 2 to 20 mm. The celiac trunk CT, the celiac plexus CP, nerve branches thereof (e.g., B1 and B2), and other neural tissue serve to innervate the pancreas P. In particular, these nerves may innervate the exocrine tissue of the pancreas P and help regulate the exocrine functions of the pancreas P.

The celiac plexus CP includes a number of smaller plexuses, such as the hepatic plexus, splenic plexus, gastric plexus, pancreatic plexus and suprarenal plexus. The celiac plexus CP is known to transmit sensation originating from the pancreas P as well as most of the abdominal viscera with the exception of the colon, rectum and pelvic organs. For example, the neurons that innervate the pancreas P can receive nociceptive stimulation and then transmit this information to the celiac plexus CP, and then to the thalamus and cortex of the brain, thereby inducing the sensation.

The present technology includes systems and methods for neuromodulation of the celiac plexus and/or celiac ganglia for efficaciously treating several clinical conditions of the abdominal viscera, such as pancreatitis and pancreatic cancer. For example, some embodiments of the treatment system 100 may include an ablation catheter configured to be positioned at or near one or more nerves innervating the pancreas. The ablation catheter may be configured to neuromodulate the targeted nerves by delivering energy to the nerves. For example, the ablation catheter of the present technology may be configured to be positioned at or near at least one of the celiac plexus CP and its smaller plexuses (such as the hepatic plexus, splenic plexus, gastric plexus, pancreatic plexus and suprarenal plexus), the celiac trunk CT, and the celiac ganglia and may be configured to deliver energy to one, some, or all of these nerves to neuromodulate the nerves. As used herein, “neuromodulation” of the celiac plexus CP and/or the celiac ganglia includes the partial or complete incapacitation or other effective disruption or regulation of nerves innervating the pancreas, e.g., nerves terminating in or originating from the pancreas or in structures closely associated with the pancreas) and/or nerves innervating the liver, gallbladder, stomach, spleen, kidney, small intestine, ascending and transverse colon and the ovarian theca, respectively. In particular, neuromodulation of the celiac plexus CP comprises inhibiting, reducing, blocking, pacing, up-regulating, and/or down-regulating neural communication along neural fibers (e.g., efferent and/or afferent neural fibers) innervating the pancreas, or in other embodiments, innervating the liver, gallbladder, and other abdominal organs.

In some embodiments, the treatment system 100 may access the nerves of one or more of the smaller plexuses within the celiac plexus CP, such as the hepatic plexus (e.g., along the hepatic artery), the splenic plexus (e.g., along the splenic artery), the gastric plexus (e.g., along the left gastric artery), and the pancreatic plexus (e.g., along the pancreatic after) via intravenous access through femoral, brachial or radial approaches where the ablation catheter is navigated through the celiac trunk CT to the subsidiary arteries (e.g., hepatic, splenic, pancreatic, etc.). Such incapacitation, disruption, and/or regulation can be long-term (e.g., permanent or for periods of months, years, or decades) or short-term (e.g., for periods of minutes, hours, days, or weeks).

Sympathetic neural activity via the nerve fibers of the celiac plexus and/or celiac ganglion, and particularly sympathetic afferent nerves, are responsible for carrying signals from the abdominal viscera to the brain in patients e.g., patients with conditions and diseases of the pancreas, including, but not limited to, acute pancreatitis, chronic pancreatitis, and pancreatic cancer. Neuromodulation of the celiac plexus and/or the celiac ganglia is expected to be useful in treating these conditions, as well as treating hepatobiliary disease and visceral artery insufficiency.

Additionally or alternatively, the treatment systems 100 of the present technology may be configured to neuromodulate the superior mesenteric plexus and/or the superior mesenteric ganglion. The superior mesenteric plexus is a continuation of the lower part of the celiac plexus CP. The superior mesenteric plexus surrounds the superior mesenteric artery SM (FIG. 16A) and divides into a number of secondary plexuses and/or gives rise to sympathetic nerve fibers innervating the pancreas, the small intestine, and colon in the abdomen. The superior mesenteric ganglion is the synapse point for one of the pre- and post-synaptic nerves of the sympathetic division of the autonomous nervous system. Specifically, contributions to the superior mesenteric ganglion arise from TV10 and TV11, and these nerve fibers go on to innervate the small intestine, the ascending colon and the transverse colon.

As used herein, “neuromodulation” of the superior mesenteric plexus and/or the superior mesenteric ganglia includes the partial or complete incapacitation or other effective disruption or regulation of nerves innervating the pancreas (e.g., nerves terminating in or originating from the pancreas or in structures closely associated with the pancreas) and/or nerves innervating the small intestine, and ascending and transverse colon. In particular, neuromodulation of the superior mesenteric plexus comprises inhibiting, reducing, blocking, pacing, up-regulating, and/or down-regulating neural communication along neural fibers (e.g., efferent and/or afferent neural fibers) innervating the pancreas, or in other embodiments, innervating the small intestine, and ascending and transverse colon. Such incapacitation, disruption, and/or regulation can be long-term (e.g., permanent or for periods of months, years, or decades) or short-term (e.g., for periods of minutes, hours, days, or weeks).

Similar to the sympathetic neural activity via the nerve fibers of the celiac plexus and/or celiac ganglion, the sympathetic neural activity associated with the superior mesenteric plexus and/or the superior mesenteric ganglia can be associated with carrying signals from the abdominal viscera to the brain in patients e.g., patients with conditions and diseases of the pancreas, including, but not limited to, acute pancreatitis, chronic pancreatitis, and pancreatic cancer. Neuromodulation of the superior mesenteric plexus and/or the superior mesenteric ganglia is expected to be useful in treating these conditions, as well as other conditions (e.g., cancer) associated with the small intestine and colon and with visceral artery insufficiency. Also as described above, neuromodulation of the superior mesenteric plexus and/or the superior mesenteric ganglia is also expected to be useful in treating clinical conditions associated with central sympathetic activity (e.g., overactivity or hyperactivity), particularly conditions associated with central sympathetic overstimulation.

### VII. Selected Intraductal Approaches to Modulating the Pancreas

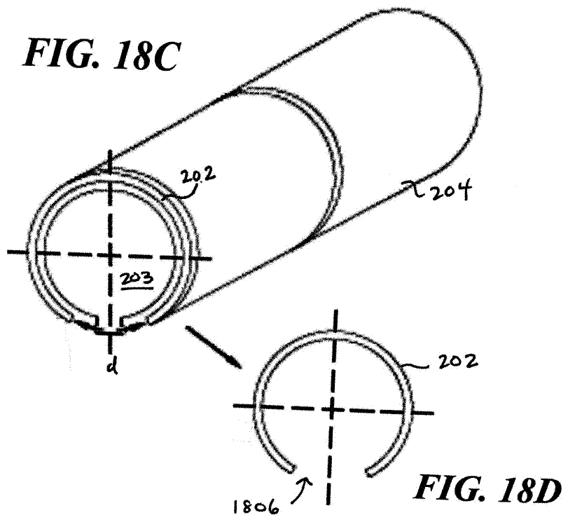

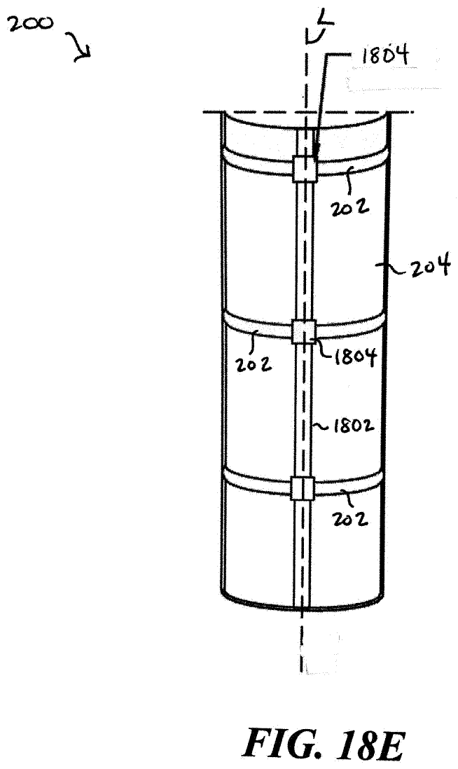

FIGS. 18A-18E illustrate a treatment device 200 for modulating retroperitoneal tissue, such as pancreatic tissue, according to several embodiments of the present technology. The treatment device 200 may include an elongated tubular shaft 204 having a distal portion 200b (FIG. 18B) configured to be positioned within the pancreatic duct and/or navigate all or a portion of the length of the pancreatic duct. For example, the shaft 204 may have an outer diameter of about 7 Fr to about 11 Fr, or about 8 Fr to about 10 Fr. The treatment device 200 may be configured to be delivered to the pancreatic duct through one or more lumens of a delivery system (such as any of the treatment systems detailed herein), or may comprise its own delivery shaft or may otherwise not require a separate delivery device.

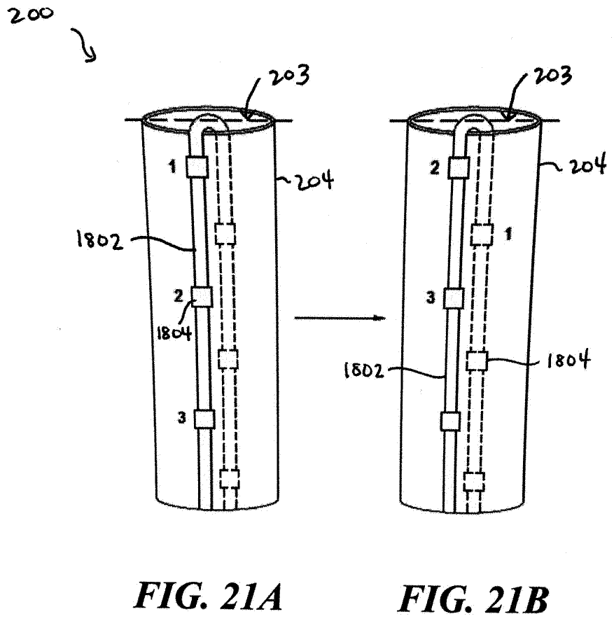

The treatment device 200 may include one or more treatment elements 202 carried by the shaft 204 and configured to be implanted at or within the pancreatic duct. In some embodiments, for example as shown in FIGS. 18A-18E, the treatment device 200 includes a plurality of treatment elements 202 configured to be detachably coupled to an exterior location of the elongated shaft 204. The treatment elements 202 may be coupled to the shaft 204 via a conveyor 1802 extending along all or a portion of the length of the shaft 204. In some embodiments, the treatment elements 202 are releasably coupled to the conveyor 1802 via a coupling portion 1804. The conveyor 1802 may be configured to slide longitudinally along the shaft 204 such that distal movement of the conveyor 1802 relative to the shaft 204 causes the attached treatment elements 202 to move distally along the exterior of the shaft 204 towards the distal terminus of the shaft 204. When a given treatment element 202 reaches the distal terminus, the conveyor 1802 and/or coupling portion 1804 releases the treatment element 202 (for example, into the lumen of the pancreatic duct). As the conveyor 1802 continues to be advanced, the conveyor 1802 inverts over the distal end of the shaft 204 then travels proximally along the shaft lumen 203, as depicted in FIGS. 21A and 21B.

In some embodiments, the treatment elements 202 may be coupled to the conveyor 1802 at preset intervals and released into the duct lumen sequentially. In some embodiments, the treatment elements 202 may be configured to be released from the shaft 204 at locations along the longitudinal axis L of the shaft 204 other than the distal terminus. Additionally or alternatively, a single treatment element 202 or multiple treatment elements 202 may be released simultaneously from the same location or different locations along the longitudinal axis L of the shaft 204.

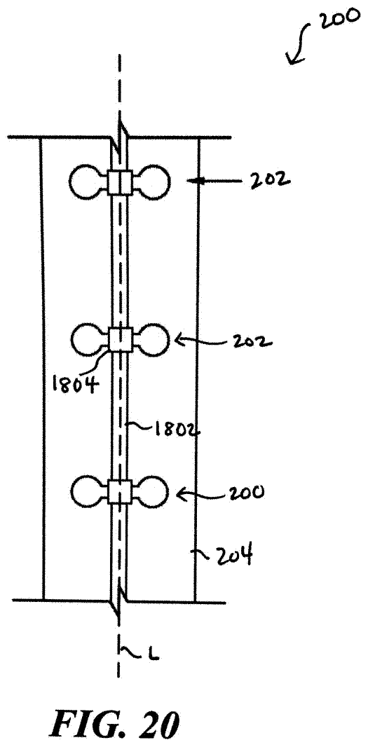

Any of the treatment elements 202 detailed herein may be configured for us with the treatment device 200 described with respect to FIGS. 18A-18E. In some embodiments, for example as shown in FIG. 18D, all or a portion of one or more of the treatment elements 202 may have a shape that that is complementary to the cross-sectional shape of the elongated shaft 204. For example, as shown in FIGS. 18A-18E, one or more of the treatment elements 202 may comprise an open ring having a gap 1806 along its circumference. The treatment element 202 may extend around and/or conform to all or a portion of the cross-sectional shape of the shaft 204. In some embodiments, the treatment element 202 may comprise a coil (as shown in FIG. 20) and/or other means for mechanically obstructing the pancreatic duct. By delivering the treatment elements 202 via an external portion of the shaft 204, the lumen 203 of the shaft 204 may be used to facilitate an adjunctive therapy such as, for example, delivery of a liquid embolic, delivery of an additional occlusive element, delivery of an additional elongated shaft, delivery of an ablation device, delivery of a suction device, etc.

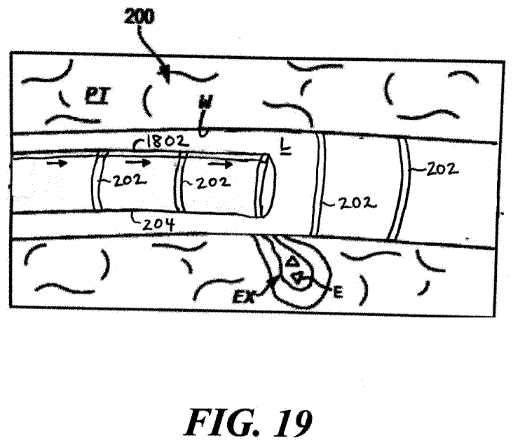

As depicted in FIG. 19, the treatment device 200 may be positioned in the pancreatic duct lumen and release one or more treatment elements 202, thereby obstructing at least a portion of the volume of the pancreatic duct. The treatment elements 202 may expand into apposition with an inner surface of the duct wall W, or may maintain generally the same shape and/or size once released from the shaft 204.

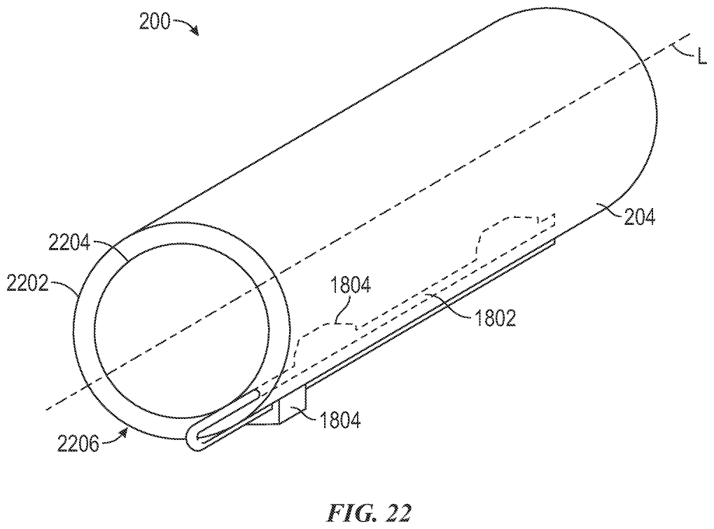

FIG. 22 depicts a treatment device 200 comprising an outer shaft 2202 and an inner shaft 2204 disposed at least partially within and surrounded by the outer shaft 2202. The outer shaft 2202 may include a conveyor system (such as conveyor 1802 and/or coupling portions 1804) configured to shuttle one or more treatment elements 202 to a distal end portion of the shaft 2204. (The treatment elements 202 are not shown in FIG. 22 for ease of viewing the outer and inner shafts 2202, 2204.) The conveyor 1802 may be positioned between the outer and inner shafts 2202, 2204. The inner shaft 2204 may be configured to facilitate an adjunctive therapy while the outer shaft 2202 positions one or more occlusive elements. The adjunctive therapy may comprise, for example, delivery of a liquid embolic, delivery of an additional occlusive element, delivery of an additional elongated shaft, delivery of an ablation device, delivery of a suction device, etc. The outer and inner shafts 2202 and 2204 may be spaced apart by a gap 2206 so that the proximally-oriented portion of the conveyor 1802 disposed within the outer shaft lumen is not impacted by the contents of the inner shaft 2202.

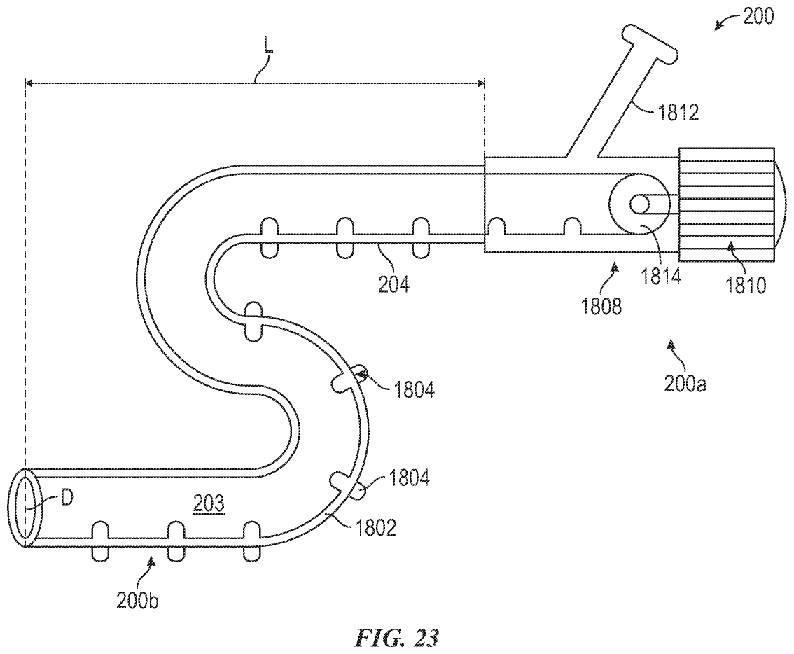

FIG. 23 is a partial cross-sectional view of a treatment device 200 having a proximal portion 200a configured to be extracorporeally positioned during treatment and a distal portion 200b configured to be endoluminally positioned at a treatment site at, within, or otherwise proximate to the pancreatic duct. The treatment device 200 may include a handle 1808 at the proximal portion and an elongated shaft 204 extending between the handle 1808 and the distal portion 200b. The elongated shaft 204 may be generally similar to the elongated shaft 204 described with reference to FIGS. 18A-18E. For example, the elongated shaft 204 may include a conveyor system (such as conveyor 1802 and/or coupling portions 1804) for transporting one or more treatment elements (not depicted) to the distal portion 200b of the shaft 204. The conveyor 1802 may extend between an actuator 1814 at the proximal portion 200a and the distal portion of the shaft 204. The actuator 1814 may be coupled to a control 1810 on the handle 1808 that allows the clinician to actuate the actuator 1814 to deliver one or more treatment elements to the treatment site. In some embodiments, the proximal portion 200a and/or handle 1808 may include one or more ports 1812 configured to be fluidly coupled to the shaft lumen 203.

The elongated shaft 204 may have a length and diameter configured to partially or fully navigate and obstruct the pancreatic duct. In some embodiments, for example, the elongated shaft 204 has a length L of about 20 cm to about 30 cm, about 22 cm to about 28 cm, about 24 cm to about 26 cm, or about 25 cm. In some embodiments, the shaft 204 may have an outer diameter D of about 7 Fr to about 11 Fr, or about 8 Fr to about 10 Fr.

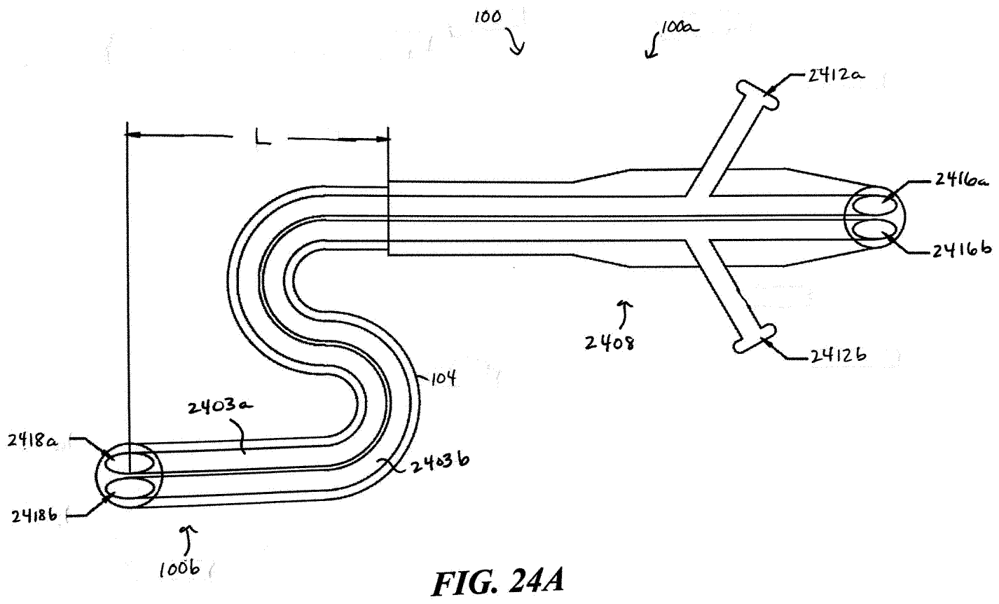

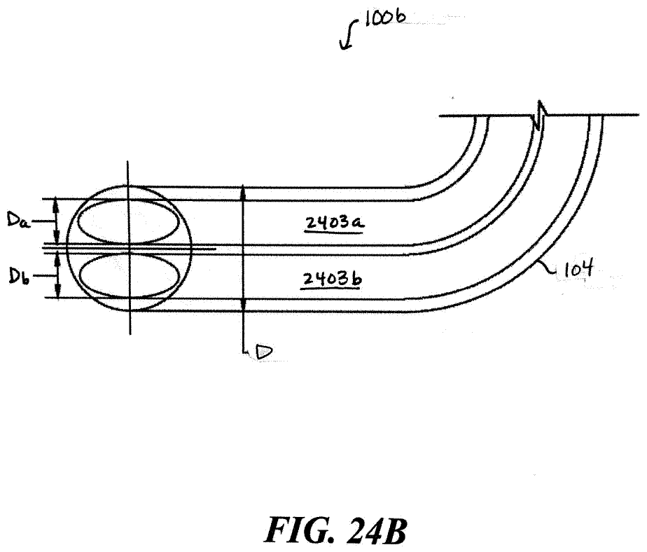

FIG. 24A is a cross-sectional view of a treatment system 100 having a proximal portion 100a configured to be extracorporeally positioned during treatment and a distal portion 100b configured to be endoluminally positioned at a treatment site at, within, or otherwise proximate to the pancreatic duct. FIG. 24B shows an enlarged view of the distal portion 100b of the treatment system 100 of FIG. 24A. Referring to FIGS. 24A and 24B together, the treatment system 100 may include a handle 2408 at the proximal portion and an elongated shaft 104 extending between the handle 2408 and the distal portion 100b. The elongated shaft 104 may comprise a generally tubular structure surrounding a first lumen 2403a and a second lumen 2403b. Such a configuration may be especially beneficial for enabling simultaneous mechanical and liquid obstruction or controlled release. The elongated shaft 104 may have a length and diameter configured to partially or fully navigate and obstruct the pancreatic duct. In some embodiments, for example, the elongated shaft 104 has a length L of about 20 cm to about 30 cm, about 22 cm to about 28 cm, about 24 cm to about 26 cm, or about 25 cm. In some embodiments, the shaft 104 may have an outer diameter D of about 7 Fr to about 11 Fr, or about 8 Fr to about 10 Fr.

The first lumen 2403a may extend between a proximal opening 2416a and a distal opening 2418a, and the second lumen 2403b may extend between a proximal opening 2416b and a distal opening 2418b. One or both of the first and second lumens 2403a, 2403b may be configured to receive a guidewire therethrough. The proximal portion 100a and/or handle 2408 may comprise one or both of a first port 2412a configured to be fluidly coupled to the first lumen 2403a and a second port 2412b configured to be fluidly coupled to the second lumen 2403b. In some embodiments, the first lumen 2403a may have a diameter of about 2 Fr to about 7 Fr, about 3 Fr to about 6 Fr, or about 4 Fr to about 5 Fr. In some embodiments, the second lumen 2403b may have a diameter of about 2 Fr to about 7 Fr, about 3 Fr to about 6 Fr, or about 4 Fr to about 5 Fr. Each of the first and second lumens 2403a, 2403b may be configured to receive one or more elongated shafts, members, interventional devices and/or treatment devices therethrough.

### CONCLUSION

Although many of the embodiments are described above with respect to systems, devices, and methods for treating the pancreas, the technology is applicable to other applications and/or other approaches, such as for obstructing one or more portions of the bile duct, a ureter, the gastrointestinal tract (such as the duodenum and colon), the liver, and the spleen. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 2-24B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for treating a medical condition affecting the pancreas of a human patient, the pancreas including a pancreatic duct having a lumen extending therethrough, the method comprising:
an elongated member having a proximal region and a distal region, the distal region configured to be endoluminally delivered to a treatment site at or within the pancreatic duct, wherein the distal region is configured to navigate the lumen of the pancreatic duct; and
an occlusion element detachably coupled to the distal region of the elongated member, the occlusion element having a collapsed configuration for endoluminal delivery to the treatment site and an expanded configuration for implantation at the treatment site, wherein the occlusion element forms an obstruction within the pancreatic duct lumen while implanted that blocks the flow of fluids into and/or through the pancreatic duct lumen.

2. The device of claim 1, wherein, when implanted in the pancreatic lumen, the occlusion element induces atrophy of pancreatic tissue.

3. The device of claim 1, wherein, when implanted in the pancreatic lumen, the occlusion element induces atrophy of pancreatic exocrine tissue.

4. The device of claim 1, wherein, when implanted in the pancreatic lumen, the occlusion element substantially prevents exocrine duct secretions from entering the pancreatic duct lumen from one or more adjacent exocrine ducts.

5. The device of claim 1, wherein, when implanted in the pancreatic lumen, the occlusion element substantially prevents exocrine duct secretions from traveling proximally beyond the treatment element.

6. The device of claim 1, wherein the occlusion element is configured to be expanded into apposition with an inner surface of the pancreatic duct wall such that the occlusion element obstructs at least a portion of the cross-sectional area of the pancreatic duct lumen while implanted.

7. The device of claim 1, wherein the occlusion element is configured to be expanded into apposition with an inner surface of the pancreatic duct wall such that the occlusion element obstructs substantially all of the cross-sectional area of the pancreatic duct lumen while implanted.

8. The device of claim 1, wherein the occlusion element is configured to be expanded into apposition with an inner surface of the pancreatic duct wall such that the occlusion element obstructs less than all of the cross-sectional area of the pancreatic duct lumen while implanted.

9. The device of claim 1, wherein the pancreatic duct is the main pancreatic duct.

10. The device of claim 1, wherein the pancreatic duct is the accessory pancreatic duct.

11. The device of claim 1, wherein, when implanted in the pancreatic duct lumen, the occlusion element extends along substantially the entire length of the main pancreatic duct, the entire length of the accessory pancreatic duct, or both.

12. The device of claim 1, wherein, when implanted in the pancreatic duct lumen, the occlusion element extends along less than the entire length of the main pancreatic duct, the entire length of the accessory pancreatic duct, or both.

13. The device of claim 1, wherein, when implanted in the pancreatic duct lumen, the occlusion element extends along less than an entire length of the main pancreatic duct, less than an entire length of the accessory pancreatic duct, or both.

14. A device for treating a medical condition affecting the pancreas of a human patient, the pancreas including a pancreatic duct having a lumen extending therethrough, the device comprising:
an elongated member having a proximal region and a distal region, the distal region configured to be endoluminally delivered to a treatment site at or within the pancreatic duct, wherein the distal region is configured to navigate the lumen of the pancreatic duct; and
an occlusion element configured to be implanted within a lumen of the pancreatic duct to block exocrine duct secretions from entering the pancreatic duct lumen and/or to prevent exocrine duct secretions from exiting the pancreatic duct.

15. The device of claim 14, wherein the occlusion element comprises an expandable mesh.

16. The device of claim 14, wherein the occlusion element comprises a liquid embolic.

17. The device of claim 14, wherein, the occlusion element comprises one or more embolic coils.

18. A system for treating a medical condition affecting the pancreas of a human patient, the pancreas including a pancreatic duct having a lumen extending therethrough, the system comprising:
an elongated shaft defining a lumen therethrough, the elongated shaft having a distal portion configured to be endoluminally positioned within the duodenum proximate a major duodenal papilla;
an elongated member configured to be slidably received within the lumen of the elongated shaft, the elongated member having a proximal portion and a distal portion, the distal portion configured to pass through the major duodenal papilla and into the pancreatic duct lumen; and
an occlusion element detachably coupled to the distal portion of the elongated member, the occlusion element configured to be implanted within the lumen of the pancreatic duct to block exocrine duct secretions from entering the pancreatic duct lumen and/or to prevent exocrine duct secretions from exiting the pancreatic duct.

19. The system of claim 18, wherein the elongated shaft is part of an endoscope.

20. The system of claim 18, further comprising an ablation element configured to be delivered through the elongated shaft to the pancreatic duct lumen, wherein the ablation element is configured to deliver energy to a wall of the pancreatic duct to create an obstruction in the form of scar tissue.
